(19) United States
(12) Patent Application Publication
(10) Pub. No.: US 2002/0061309 A1
(43) Pub. Date: May 23, 2002

(54) Production of peptides in plants as N-terminal viral coat protein fusions

(76) Inventors: Stephen J. Garger, Vacaville, CA (US); Cynthia Gross, Vacaville, CA (US); John A. Lindbo, Vacaville, CA (US); Gregory P. Pogue, Vacaville, CA (US)

Correspondence Address: HOWREY SIMON ARNOLD & WHITE, LLP, BOX 34, 301 RAVENSWOOD AVE., MENLO PARK, CA, 94025, US

(21) Appl. No.: 09/823,936

(22) Filed: Mar. 29, 2001

Related U.S. Application Data
(63) Continuation-in-part of application No. 09/520,967, filed on Mar. 8, 2000.

(51) Int. Cl. C12N 7/01; A01H 5/00; A61K 39/12; C07H 21/04; A61K 38/04; A61K 39/38

(52) U.S. Cl. 424/184.1; 435/235.1; 800/288; 530/326; 536/23.72

(57) ABSTRACT

The present invention relates to foreign peptide sequences fused to the N-terminal of plant viral structural proteins and a method of their production. Fusion proteins are economically synthesized in plants at high levels by biologically contained tobamoviruses. The foreign peptide sequences can be cleaved from the fusion proteins by proteolytic enzymes or chemical reagents. The foreign peptide sequences of the invention have many uses. Such uses include use as antigens for inducing the production of antibodies having desired binding properties, e.g., protective antibodies, for use as vaccine antigens for the induction of protective immunity, including immunity against parasitic infections, for use as a protein involved in hormonal activity, or for use as a protein involved in immunoregulatory activity.

### CROSS-REFERENCE TO RELATED APPLICATIONS

The present application is a continuation-in-part of U.S. patent application Ser. No. 09/520,967, filed Mar. 8, 2000.

### FIELD OF THE INVENTION

The present invention relates to the field of genetically engineered peptide production in plants, more specifically, the invention relates to the use of tobamovirus vectors to express fusion proteins, the process of expressing fusion proteins from such tobamovirus vectors, and cleaving the protein fusions.

### BACKGROUND OF THE INVENTION

Peptides are a diverse class of molecules having a variety of important chemical and biological properties. Some examples include; hormones, cytokines, immunoregulators, peptide-based enzyme inhibitors, vaccine antigens, adhesions, receptor binding domains, enzyme inhibitors and the like. The cost of chemical synthesis limits the potential applications of synthetic peptides for many useful purposes such as large scale therapeutic drug or vaccine synthesis. There is a need for inexpensive and rapid synthesis of milligram and larger quantities of naturally-occurring polypeptides. Towards this goal many animal and bacterial viruses have been successfully used as peptide carriers.

The safe and inexpensive culture of plants provides an improved alternative host for the cost-effective production of such peptides. During the last decade, considerable progress has been made in expressing foreign genes in plants. Foreign proteins are now routinely produced in many plant species for modification of the plant or for production of proteins for use after extraction. Animal proteins have been effectively produced in plants (reviewed in Krebbers et al., 1992). Having foreign proteins synthesized in plants also have the added advantage that these plant-synthesized foreign proteins are obtained in a form that is relatively free of both bacterial-related toxins and organisms or particles pathogenic to humans.

Vectors for the genetic manipulation of plants have been derived from several naturally occurring plant viruses, including TMV. TMV is the type member of the tobamovirus group. TMV has straight tubular virions of approximately 300×18 nm with a 4 nm-diameter hollow canal, consisting of approximately 2000 units of a single capsid protein wound helically around a single RNA molecule. Virion particles are 95% protein and 5% RNA by weight. The genome of TMV is composed of a single-stranded RNA of 6395 nucleotides containing five large ORFs. Expression of each gene is regulated independently. The virion RNA serves as the messenger RNA (mRNA) for the 5′ genes, encoding the 126 kDa replicase subunit and the overlapping 183 kDa replicase subunit that is produced by read through of an amber stop codon approximately 5% of the time. Expression of the internal genes is controlled by different promoters on the minus-sense RNA that direct synthesis of 3′-coterminal subgenomic mRNAs which are produced during replication (FIG. 1). A detailed description of tobamovirus gene expression and life cycle can be found, among other places, in Dawson and Lehto, Advances in Virus Research 38:307-42 (1991). It is of interest to provide new and improved vectors for the genetic manipulation of plants.

For production of specific proteins, transient expression of foreign genes in plants using virus-based vectors has several advantages. Products of plant viruses are among the highest produced proteins in plants. Often a viral gene product is the major protein produced in plant cells during virus replication. Many viruses are able to quickly move from an initial infection site to almost all cells of the plant. Because of these reasons, plant viruses have been developed into efficient transient expression vectors for foreign genes in plants. Viruses of multicellular plants are relatively small, probably due to the size limitation in the pathways that allow viruses to move to adjacent cells in the systemic infection of entire plants. Most plant viruses have single-stranded RNA genomes of less than 10 kb. Genetically altered plant viruses provide one efficient means of transfecting plants with genes coding for peptide carrier fusions. A discussion of TMV coat protein fusions is provided in Turpen et al., U.S. Pat. No. 5,977,438 entitled “Production of Peptides in Plants as Viral Coat Protein Fusions”. See also, Yusibov V., et al., Proc. Natl. Acad. Sci. USA 94:5784-88 (1997); Modelska, A, et al., Proc. Natl. Acad. Sci. USA 95:2481-85 (1998).

The pathogenesis of parvovirus infection has been most recently reviewed by Parish, C. R., Baillieres Clin. Haematol. 8:57-71 (1995). Feline parvovirus (FPV) is closely related to canine parvovirus and the respective diseases are similar in pathogenesis. Parvovirus replicates first in the tonsils, and then spreads to its target cells: mitotically active intestinal crypt epithelial cells and bone marrow stem cells. Viremia lasts for less than 7 days before death or recovery. Clinical signs in cats include fever, vomiting, diarrhea, panleukopenia, acute shock and death. The disease outcome is proportional to the severity of the leukopenia; cats with severe panleukopenia will often die, while those with mild leukopenia will usually survive. The VP2 epitope of mink enteritis virus (MEV), which is closely related to FPV, has been previously expressed on the surface of cowpea mosaic virus, which was propagated on the leaves of the black-eyed bean (Dalsgaard, K et al., Nature Biotechnol. 15:248-52 (1997)). One mg of the cow pea mosaic virus material that expressed this epitope was used to immunize minks against virulent MEV. The minks were protected against clinical disease, and shed very little virus. The authors suggested that this epitope, expressed in this manner, could also be used to protect cats and dogs against their respective parvovirus infections. The coding sequence for VP2 (E2) and the rabies spike glycoprotein have also been engineered into raccoon poxvirus to make a five recombinant vaccine against rabies and feline panleukopenia (Hu, L. et al., Virol. 218:248-52 (1996); Hu, L. et al., Vaccine 15: 1466-72 (1997)). Cats vaccinated with this construct showed excellent protection against virulent parvovirus challenge.

The present invention provides polynucleotides that encode fusion proteins comprising a protein of interest linked to the N-terminal of a plant viral coat protein (“VCP”) via a linking element. The present invention also provides methods for the production of the protein of interest using the subject polynucleotides and fusion proteins. An advantage of the N-terminus fusion protein of the present invention is that it allows viral assembly and this leads to the ability for one of ordinary skill in the art to purify intact viral particles. Another advantage of the invention is that the protein of interest is cleavable from the fusion protein using a cleaving agent. Another advantage is that the fusion protein is capable of being extracted using methods that are scalable and the subsequent cleavage of the protein of interest from the fusion protein is also scalable. Another advantage is that initiation of translation from an internal methionine may result in the expression of wild-type plant VCP which would result in a higher production of viral particles that would result in viral properties that facilitate virus extraction and purification.

### SUMMARY OF THE INVENTION

The present invention provides polynucleotides that encode fusion proteins that comprise fusions between a plant VCP and a protein of interest at the N-terminal of the plant VCP via a linking element. A second protein of interest may be fused to the fusion protein at the carboxyl terminal of the plant VCP or internal to the plant VCP. By infecting plant cells with the recombinant plant viruses of the invention, relatively large quantities of the protein of interest may be produced in the form of a fusion protein. The protein of interest can be isolated from the plant VCP by cleaving the protein of interest from the plant VCP by a chemical or catalytic means using a catalytic agent that cleaves a covalent bond within the linking element. The fusion protein encoded by the recombinant plant virus may have any of a variety of forms. The fusion protein may have one or more properties of the protein of interest. The fusion protein may have one or more properties of the second protein of interest. The isolated protein of interest or the fusion protein may be used as an antigen for antibody development or to induce a protective immune response. The present invention also encompasses methods for synthesizing the protein of interest by expressing the fusion protein using the polynucleotide and optionally cleaving the protein of interest portion from the fusion protein.

Another aspect of the invention is to provide recombinant viral nucleic acids, recombinant viral genome, recombinant virus particles, recombinant plant viruses, plants, plant cells, plant protoplasts, and the like that comprise such polynucleotides. Another aspect of the invention is to provide polynucleotides encoding the genomes of the subject recombinant plant viruses. Another aspect of the invention is to provide the fusion proteins encoded by the subject recombinant plant viruses. Yet another embodiment of the invention is to provide plant cells that have been infected by the recombinant plant viruses of the invention.

The invention also provides for methods for the synthesizing of the protein of interest by expressing the subject fusion protein using the subject polynucleotide.

### BRIEF DESCRIPTION OF THE FIGURES

FIG. 1 depicts a Tobamovirus Gene Expression. The gene expression of tobamoviruses is diagrammed.

FIG. 2 depicts a plasmid map of the TMV Transcription Vector pSNC004. The infectious RNA genome of the U1 strain of TMV is synthesized by T7 RNA polymerase in vitro from pSNC004 linearized with KpnI.

FIG. 3 depicts a diagram of plasmid constructions. Each step in the construction of plasmid DNAs encoding various viral epitope fusion vectors discussed in the examples is diagrammed.

FIG. 4 depicts the binding of monoclonal antibody (NVS3) to TMV291. The reactivity of NVS3 to the malaria epitope present in TMV291 is measured in a standard ELISA.

FIG. 5 depicts the binding of monoclonal antibody (NYS1) to TMV261. The reactivity of NYS1 to the malaria epitope present in TMV261 is measured in a standard ELISA.

FIG. 6 depicts the location of oligos and restriction sites used in the construction of pJL150/198 and pJL150/199.

FIG. 7 depicts the MALDI-TOF analysis of PEG purified virion preparations derived from Supernatant 1 of Example 15 (see Table 6). The peaks of three protein masses were detected corresponding to the predicted fall length TMV coat-peptide fusion (indicated by 19,163), a proteolytic degradation product containing an N-terminal arginine residue (indicated by 18,104), and a protein containing an N-terminal methionine residue resulting initiation of translation on an internal methionine or proteolytic degradation (17,537).

FIG. 8 depicts the MALDI-TOF analysis of PEG purified virion preparations derived from Supernatant 2 of Example 15 (see Table 6). The peaks of three protein masses were detected corresponding to the predicted full length TMV coat-peptide fusion (indicated by 19,188), a proteolytic degradation product containing an N-terminal arginine residue (indicated by 18,121), and a protein containing an N-terminal methionine residue resulting initiation of translation on an internal methionine or proteolytic degradation (17,550).

FIG. 9 depicts the MALDI-TOF analysis of cyanogen bromide cleaved products in Example 16 (see Table 6). The CNBr pellet contained two products with mass weights of 19,330 and 17,570 daltons corresponding to uncleaved and cleaved TMV coat, respectively. Both TMV coat species have an apparent increase in mass that is likely due to acid ester formation.

FIG. 10 depicts the MALDI-TOF analysis of the resuspended permeate lyophilisate of Example 16 (see Table 6). The resuspended 10 Kd permeate lyophilisate sample contained predominantly a 1736 dalton species and minor quantities of 1720, 1758 and 1828 dalton species. The 1736 fragment corresponds to the predicted mass of the released parvo peptide sequence containing a carboxy-terminal homoserine.

FIG. 11 depicts the MALDI-TOF analysis of the resuspended permeate lyophilisate of Example 16 (see Table 6). The resuspended 10 kDa permeate lyophilisate sample did not contain detectable amounts of uncleaved or cleaved TMV coat.

FIG. 12 depicts the HPLC chromatograms of Pellet 1 of Example 19 (see Table 6).

FIG. 13 depicts the HPLC chromatograms of Supernatant 1 of Example 19 (see Table 6).

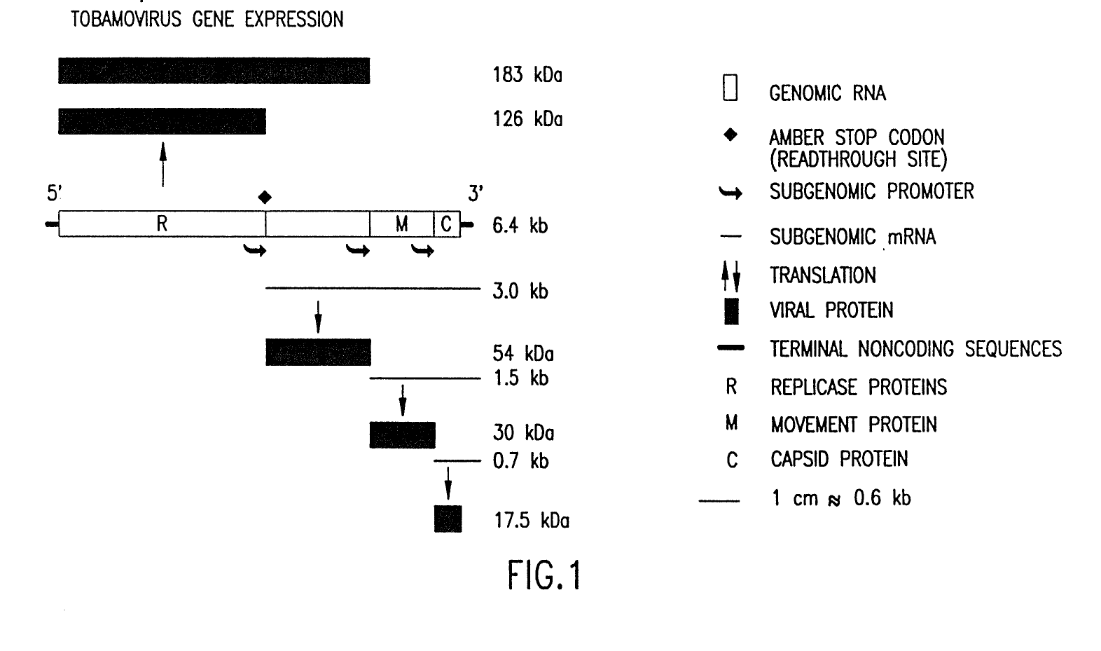

### DESCRIPTION OF THE SPECIFIC EMBODIMENTS

Definitions and Abbreviations

Cell Culture: A proliferating group of cells which may be in either an undifferentiated or differentiated state, growing contiguously or non-contiguously.

CNBr: Cyanogen bromide.

Coding Sequence: A deoxyribonucleotide or ribonucleotide sequence which, when either transcribed and translated or simply translated, results in the formation of a cellular polypeptide or a ribonucleotide sequence which, when translated, results in the formation of a cellular polypeptide.

CP: Coat protein.

ELISA: Enzyme-linked immunosorbent assay.

Expression: The term as used herein is meant to incorporate one or more of transcription, reverse transcription and translation.

FPV: Feline panleukopenia virus.

Gene: A discrete nucleic acid sequence responsible for producing one or more cellular products and/or performing one or more intercellular or intracellular functions.

HPLC: High Performance Liquid Chromatography.

Infection: The ability of a virus to transfer its nucleic acid to a host or introduce a viral nucleic acid into a host, wherein the viral nucleic acid is replicated, viral proteins are synthesized, and new viral particles assembled. In this context, the terms “transmissible” and “infective” are used interchangeably herein. The term is also meant to include the ability of a selected nucleic acid sequence to integrate into a genome, chromosome or gene of a target organism.

MALDI-TOF: Matrix-assisted laser desorption time of flight mass spectrometry.

Nucleic acid: As used herein the term is meant to include any DNA or RNA sequence from the size of one or more nucleotides up to and including a complete gene sequence. The term is intended to encompass all nucleic acids whether naturally occurring in a particular cell or organism or non-naturally occurring in a particular cell or organism.

ORF: Open reading frame.

PAGE: Polyacrylamide gel electrophoresis.

PCR: Polymerase chain reaction.

PEG: Polyethylene glycol.

PEI: Polyethyleneimine.

Plant Cell: The structural and physiological unit of plants, consisting of a protoplast and the cell wall.

Promoter: The 5′-flanking, non-coding sequence substantially adjacent a coding sequence which is involved in the initiation of transcription of the coding sequence.

Protoplast: An isolated plant or bacterial cell without some or all of its cell wall.

Recombinant Viral Nucleic Acid: Viral nucleic acid which has been modified to contain non-native nucleic acid sequences. These non-native nucleic acid sequences may be from any organism or purely synthetic, however, they may also include nucleic acid sequences naturally occurring in the organism into which the recombinant viral nucleic acid is to be introduced.

Recombinant Virus: A virus containing the recombinant viral nucleic acid.

Subgenomic Promoter: A promoter of a subgenomic mRNA of a viral nucleic acid.

Systemic Infection: Denotes infection throughout a substantial part of an organism including mechanisms of spread other than mere direct cell inoculation but rather including transport from one infected cell to additional cells either nearby or distant.

TMV: Tobacco mosaic tobamovirus.

TMVCP: Tobacco mosaic tobamovirus coat protein.

VCP: Viral coat protein.

Viral Particles: High molecular weight aggregates of viral structural proteins with or without genomic nucleic acids

Virion: An infectious viral particle.

The Invention

The subject invention provides novel recombinant plant viruses that code for the expression of fusion proteins that consist of a fusion between a plant VCP and a protein of interest wherein the protein of interest is fused to the N-terminus of the plant VCP via a linking element. The recombinant plant viruses of the invention provide for systemic expression of the fusion protein, by systemically infecting cells in a plant. The invention also provides for the isolation of the protein of interest from the fusion protein by the cleavage of the linking element by a cleavage agent. Thus by employing the recombinant plant viruses of the invention, large quantities of a protein of interest, either fused to the plant VCP or isolated from the fusion protein may be produced.

The fusion proteins of the invention comprise three portions: (1) a plant VCP, (2) a linking element, and (3) a protein of interest. The fusion protein may also comprise a second protein of interest. The plant VCP portion may be derived from the same plant VCP that serves a CP for the virus from which the genome of the expression vector is primarily derived, i.e., the VCP is native with respect to the recombinant viral genome. Alternatively, the VCP portion of the fusion protein may be heterologous, i.e., non-native, with respect to the recombinant viral genome. Alternatively, the plant VCP portion may be not identical to the wild-type or other natural occurring plant VCP; in such instances, the plant VCP portion, though not wild-type or naturally occurring, essentially exhibits substantially most of the biological/chemical characteristics of the wild-type or naturally occurring plant VCPs necessary for practice of the invention. In a preferred embodiment of the invention, the polynucleotide encodes a fusion protein that permits the expression of the fusion protein and the expression of the plant VCP encoded within the fusion protein. The expression of the plant VCP may be from the internal initiation of the portion of the polynucleotide or a mRNA derived from the polynucleotide encoding the plant VCP during translation or post-translation modification of the fusion protein. A post-translation modification of the fusion protein that gives rise to the plant VCP is the proteolysis of fusion protein. In a preferred embodiment of the invention, the 17.5 KDa CP of TMV is used in conjunction with a TMV derived vector.

The linking element comprises a covalent bond or a peptide of virtually any amino acid sequence, provided the linking element is specifically cleaved by the breaking of at least one covalent bond by a cleaving agent resulting in the isolation of the plant VCP portion and the protein of interest portion, i.e. the physical separation of the plant VCP portion and the protein of interest portion. The cleaving agent may consist of either a protein or non-protein that is capable of breaking one covalent bond. Examples of such proteins are trypsin, chymptrypsin, pepsin, Staphylococcus aureus V8 protease, and Factor Xa protease. An example of a non-protein is a chemical reagent such as cyanogen bromide (“CNBr”). These examples are provided to merely illustrate and do not limit the possible cleaving agents. In a preferred embodiment of the invention, a linking element comprises one or more specific amino acids that is capable of being cleaved by a specific cleaving agent. For example, if trypsin is used as a cleaving agent then the linking element would comprise a lysine or arginine residue. For example, if chymotrypsin is used as a cleaving agent then the linking element would comprise a phenylalanine, tryptophan, or tyrosine residue. For example, if pepsin is used as a cleaving agent then the linking element would comprise a phenylalanine, tryptophan, tyrosine, leucine, aspartate, or glutamate residue. For example, if S. aureus V8 protease is used as a cleaving agent then the linking element would comprise an aspartate or glutamate residue (S. aureus V8 protease cleaves at the carboxylic side of glutamate in 50 mM ammonium bicarbonate (pH 7.8), or 50 mM ammonium acetate (pH 4.0); and cleaves at the carboxylic side of both aspartate or glutamate in 50 mM sodium phosphate buffer (pH 7.8)). For example, if Factor Xa protease is used as a cleaving agent then the linking element would comprise a four residue sequence of isoleucine-glutamate-glycine-arginine. For example, if CNBr is used as a cleaving agent then the linking element would comprise a methionine residue. These examples are provided to merely illustrate and do not limit the possible cleaving agent and linking element combinations. In a preferred embodiment of the invention, one covalent bond broken is a peptide bond.

The protein of interest portion of the fusion protein for expression may consist of a peptide of virtually any amino acid sequence, provided that the protein of interest does not significantly interfere with (1) the ability to bind to a receptor molecule, including antibodies and T cell receptor (2) the ability to bind to the active site of an enzyme (3) the ability to induce an immune response, (4) hormonal activity, (5) immunoregulatory activity, and (6) metal chelating activity. The protein of interest portion of the subject fusion proteins may also possess additional chemical or biological properties that have not been enumerated. Protein of interest portions of the subject fusion proteins having the desired properties may be obtained by employing all or part of the amino acid residue sequence of a protein known to have the desired properties. For example, the amino acid sequence of hepatitis B surface antigen may be used as a protein of interest portion of a fusion protein invention so as to produce a fusion protein that has antigenic properties similar to hepatitis B surface antigen. Detailed structural and functional information about many proteins of interest are well known, this information may be used by the person of ordinary skill in the art so as to provide for fusion proteins having the desired properties of the protein of interest. The protein of interest portion of the subject fusion proteins may vary in size from one amino acid residue to over several hundred amino acid residues. The sequence of the protein of interest portion of the subject fusion protein may be from one to 100 amino acid residues in size, or from one to 50 amino acid residues in length, or from one to 25 amino acid residues in length.

While the protein of interest portion of fusion proteins of the invention may be derived or obtained from any of the variety of proteins, proteins for use as antigens are particularly preferred. For example, the fusion protein, or a portion thereof, may be injected into a mammal, along with suitable adjutants, so as to produce an immune response directed against the protein of interest portion of the fusion protein. The immune response against the protein of interest portion of the fusion protein has numerous uses, such uses include, protection against infection, and the generation of antibodies useful in immunoassays.

The fusion protein of the invention may also have a second protein of interest, other than the protein of interest and the linking element, fused to the fusion protein at a position other than the N-terminal. The location (locations) in the fusion protein of the invention where the VCP portion is joined to the second protein of interest is referred to herein as the fusion joint. A given fusion protein may have one or two fusion joints. The fusion joint may be located at the carboxy terminus of the VCP portion of the fusion protein (joined at the amino terminus of the second protein of interest portion). In other embodiments of the invention, the fusion protein may have two fusion joints. In those fusion proteins having two fusion joints, the second protein of interest is located internal with respect to the carboxyl and amino terminal amino acid residues of the VCP portion of the fusion protein, i.e., an internal fusion protein. Internal fusion proteins may comprise an entire plant VCP amino acid residue sequence, or a portion thereof, that is “interrupted” by a second protein of interest, i.e., the amino terminal segment of the VCP portion is joined at a fusion joint to the amino terminal amino acid residue of the second protein of interest and the carboxyl terminal segment of the VCP is joined at a fusion joint to the amino terminal acid residue of the second protein of interest.

When the fusion protein for expression is an internal fusion protein, the fusion joints may be located at a variety of sites within a coat protein. Suitable sites for the fusion joints may be determined either through routine systematic variation of the fusion joint locations so as to obtain an internal fusion protein with the desired properties. Suitable sites for the fusion jointly may also be determined by analysis of the three dimensional structure of the coat protein so as to determine sites for “insertion” of the protein of interest that do not significantly interfere with the structural and biological functions of the VCP portion of the fusion protein. Detailed three dimensional structures of plant VCPs and their orientation in the virus have been determined and are publicly available to a person of ordinary skill in the art. For example, a resolution model of the coat protein of Cucumber Green Mottle Mosaic Virus (a coat protein bearing strong structural similarities to other tobamovirus coat proteins) and the virus can be found in Wang et al., J. Mol. Biol. 239:371-84 (1994). Detailed structural information on the virus and CP of TMV can be found, among other places, in Namba et al., J. Mol. Biol. 208:307-25 (1989) and Pattanayek et al., J. Mol. Biol. 228:516-28 (1992).

Knowledge of the three dimensional structure of a plant virus particle and the assembly process of the virus particle permits the design of various CP fusions of the invention, including insertions, and partial substitutions. For example, if the protein of interest is of a hydrophilic nature, it may be appropriate to fuse the peptide to the TMVCP region known to be oriented as a surface loop region. Likewise, alpha helical segments that maintain subunit contacts might be substituted for appropriate regions of the TMVCP helices or nucleic acid binding domains expressed in the region of the TMVCP oriented towards the genome.

Polynucleotide sequences encoding the subject fusion proteins may comprise a “leaky” stop codon at a fusion joint. The stop codon may be present as the codon immediately adjacent to the fusion joint, or may be located close (e.g., within 9 bases) to the fusion joint. A leaky stop codon may be included in polynucleotides encoding the subject fusion proteins so as to maintain a desired ratio of fusion protein to wild-type CP. A “leaky” stop codon does not always result in translational termination and is periodically translated. The frequency of initiation or termination at a given start/stop codon is context dependent. The ribosome scans from the 5′-end of a messenger RNA for the first ATG codon. If it is in a non-optimal sequence context, the ribosome will pass, some fraction of the time, to the next available start codon and initiate translation downstream of the first. Similarly, the first termination codon encountered during translation will not function 100% of the time if it is in a particular sequence context. Consequently, many naturally occurring proteins are known to exist as a population having heterogeneous N and/or C terminal extensions. Thus by including a leaky stop codon at a fusion joint coding region in a recombinant viral vector encoding a fusion protein, the vector may be used to produce both a fusion protein and a second smaller protein, e.g., the VCP. A leaky stop codon may be used at, or proximal to, the fusion joints of fusion proteins in which the second protein of interest portion is joined to the carboxyl terminus of the CP region, whereby a single recombinant viral vector may produce both fusion proteins and CPs. Additionally, a leaky start codon may be used at or proximal to the fusion joints of fusion proteins in which the protein of interest portion is joined to the amino terminus of the coat protein region, whereby a similar result is achieved. In the case of TMVCP, extensions at the N and C terminus are at the surface of viral particles and can be expected to project away from the helical axis. An example of a leaky stop sequence occurs at the junction of the 126/183 kDa reading frames of TMV and was described over 15 years ago (Pelham, 1978). Skuzeski et al. (1991) defined necessary 3′ context requirements of this region to confer leakiness of termination on a heterologous protein marker gene (β-glucuronidase) as CAR-YYA (C=cytosine, A=adenine, R=purine, Y=pyrimidine).

In another embodiment of the invention, the fusion joints on the subject fusion proteins are designed so as to comprise an amino acid sequence that is a substrate for protease. By providing a fusion protein having such a fusion joint, the second protein of interest may be conveniently derived from the fusion protein by using a suitable proteolytic enzyme. The proteolytic enzyme may contact the fusion protein either in vitro or in vivo.

The expression of the subject fusion proteins may be driven by any of a variety of promoters functional in the genome of the recombinant plant viral vector. The subject fusion protein may also be expressed by any promoter functional in a plant or a cell 5′ to the fusion protein encoding region. In a preferred embodiment, the cell is a plant cell, a plant protoplast, a cell in a plant cell culture, or any appropriate cell. The promoter may be any viral promoter or RNA viral promoter. In a preferred embodiment, the promoter is a promoter of a single-stranded plus-sense RNA virus. In a more preferred embodiment, the promoter is a promoter of a tobamovirus. In an even more preferred embodiment, the promoter is a promoter of a TMV. In an even further more preferred embodiment, the promoter is the promoter of the CP gene of TMV. In another preferred embodiment of the invention, the subject fusion proteins are expressed from plant viral subgenomic promoters using vectors as described in U.S. Pat. No. 5,316,931. The expression of the subject fusion protein may be elevated or controlled by a variety of plant or viral transcription factors.

In another embodiment of the invention, the fusion protein has an internal methionine, or any other amino acid capable of initiating translation, near the N-terminal of the fusion protein whereby initiation of translation can take place. The initiation of translation from such an internal methionine, or any other amino acid capable of initiating translation, results in the expression of a wild-type CP or peptide that results in a higher number of viral particles assembled then if no internal initiation of the wild-type CP or peptide took place. In one embodiment, the internal methionine, or any other amino acid capable of initiating translation, is an amino acid of the linking element of the fusion protein. In a preferred embodiment, the internal methionine, or any other amino acid capable of initiating translation, is less than 100 amino acids residues from the first amino acid residue of the fusion protein. In a more preferred embodiment, the internal methionine, or any other amino acid capable of initiating translation, is less than 50 amino acids residues from the first amino acid residue of the fusion protein. In an even more preferred embodiment, the internal methionine, or any other amino acid capable of initiating translation, is less than 25 amino acids residues from the first amino acid residue of the fusion protein. In an even further more preferred embodiment, the internal methionine, or any other amino acid capable of initiating translation, is less than 20 amino acids residues from the first amino acid residue of the fusion protein. In another embodiment, the internal methionine is capable of being cleaved by CNBr.

Recombinant DNA technologies have allowed the life cycle of numerous plant RNA viruses to be extended artificially through a DNA phase that facilitates manipulation of the viral genome. These techniques may be applied by the person ordinary skill in the art in order make and use recombinant plant viruses of the invention. The entire cDNA of the TMV genome was cloned and functionally joined to a bacterial promoter in an E. coli plasmid (Dawson, et al., 1986). Infectious recombinant plant viral RNA transcripts may also be produced using other well known techniques, for example, with the commercially available RNA polymerases from T7, T3 or SP6. Precise replicas of the virion RNA can be produced in vitro with RNA polymerase and dinucleotide cap, m7GpppG. This not only allows manipulation of the viral genome for reverse genetics, but it also allows manipulation of the virus into a vector to express foreign genes. A method of producing plant RNA virus vectors based on manipulating RNA fragments with RNA ligase has proved to be impractical and is not widely used (Pelcher, 1982). Detailed information on how to make and use recombinant RNA plant viruses can be found, among other places in U.S. Pat. No. 5,316,931 (Donson, et al.), which is herein incorporated by reference. The invention provides for polynucleotide encoding recombinant RNA plant vectors for the expression of the subject fusion proteins. The invention also provides for polynucleotides comprising a portion or portions of the subject vectors. The vectors described in U.S. Pat. No. 5,316,931 are particularly preferred for expressing the fusion proteins of the invention.

In addition to providing the described fusion proteins, the invention also provides for virus particles that comprise the subject polynucleotide. The coat of the virus particles of the invention may consist entirely of fusion protein encoded by the subject polynucleotide. In another embodiment of the virus particles of the invention, the virus particle coat may consist of a mixture of fusion proteins and non-fusion CP, wherein the ratio of the two proteins may be varied. As tobamovirus coat proteins may self-assemble into virus particles, the virus particles of the invention may be assembled either in vivo or in vitro. The virus particles may also be conveniently disassembled using well known techniques so as to simplify the purification of the subject fusion proteins, or portions thereof.

The invention also provides for recombinant plant cells, plant cells, plant protoplasts, and the like comprising the subject fusion proteins and/or virus particles comprising the subject fusion proteins. These cells may be produced either by infecting cells (either in culture or in whole plants) with infections virus particles of the invention or with polynucleotides encoding the genomes of the infectious virus particle of the invention. The cells have many uses, for example, serving as a source or site for expression of the fusion proteins of the invention.

The protein of interest portion of the subject fusion proteins may comprise many different amino acid residue sequences, and accordingly many different possible biological/chemical properties however, in a preferred embodiment of the invention the protein of interest portion of the fusion protein, and/or the second protein of interest, is useful as a vaccine antigen. The surface of TMV particles and other tobamoviruses contain continuous epitopes of high antigenicity and segmental mobility thereby making TMV particles especially useful in producing a desired immune response. These properties make the virus particles of the invention especially useful as carriers in the presentation of foreign epitopes to mammalian immune systems.

The recombinant RNA viruses of the invention may be used to produce numerous fusion proteins for use as vaccine antigens or vaccine antigen precursors. One vaccine of interest is that against malaria. Human malaria is caused by the protozoan species Plasmodium falciparum, P. vivax, P. ovale and P. malariae and is transmitted in the sporozoite form by Anopheles mosquitos. Control of this disease will likely require safe and stable vaccines. Several peptide epitopes expressed during various stages of the parasite life cycle are thought to contribute to the induction of protective immunity in partially resistant individuals living in endemic areas and in individuals experimentally immunized with irradiated sporozoites.

When the fusion proteins of the invention, portions thereof, or viral particles comprising the fusion proteins are used in vivo, the proteins are typically administered in a composition comprising a pharmaceutical carrier. A pharmaceutical carrier can be any compatible, non-toxic substance suitable for delivery of the desired compounds to the body. Sterile water, alcohol, fats, waxes and inert solids may be included in the carrier. Pharmaceutically accepted adjuvants (buffering agents, dispersing agent) may also be incorporated into the pharmaceutical composition. Additionally, when the subject protein of interest, or the subject fusion proteins, or portion thereof, are to be used for the generation of an immune response, protective or otherwise, formulation for administration may comprise one or immunological adjuvants in order to stimulate a desired immune response.

When the protein of interest, the second protein of interest or the fusion protein of the invention, or portions thereof, are used in vivo, they may be administered to a subject, human or animal, in a variety of ways. The pharmaceutical compositions may be administered orally or parenterally, i.e., subcutaneously, intramuscularly or intravenously. Thus, this invention provides compositions for parenteral administration which comprise a solution of the protein of interest or the fusion protein, or derivative thereof, or a cocktail thereof dissolved in an acceptable carrier, preferably an aqueous carrier. A variety of aqueous carriers can be used, e.g., water, buffered water, 0.4% saline, 0.3% glycerine and the like. These solutions are sterile and generally free of particulate matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example sodium acetate, sodium chloride, potassium chloride, calcium chloride, sodium lactate, etc. The concentration of protein of interest or fusion protein (or portion thereof) in these formulations can vary widely depending on the specific amino acid sequence of the subject proteins and the desired biological activity, e.g., from less than about 0.5%, usually at or at least about 1% to as much as 15 or 20% by weight and will be selected primarily based on fluid volumes, viscosities, etc., in accordance with the particular mode of administration selected. Actual methods for preparing parenterally administrable compositions and adjustments necessary for administration to subjects will be known or apparent to those skilled in the art and are described in more detail in, for example, Remington's Pharmaceutical Science, current edition, Mack Publishing Co., Easton, Pa., which is incorporated herein by reference.

The invention also encompasses methods for the synthesizing the protein of interest by expressing the subject fusion protein using the subject polynucleotide. The method comprises contacting a plant or a plant cell with a recombinant plant viral nucleic acid comprising the subject polynucleotide; and growing, expanding or cultivating the plant or the plant cell under conditions such that the fusion protein is expressed. The plant cell may be a plant protoplast, or a cell of a plant cell culture, or any appropriate cell. The method may further comprise reacting the linking element with a cleaving agent, either in vitro or in vivo, such that one covalent bond between the protein of interest and the plant VCP is broken. The covalent bond is preferably a peptide bond. The method may further comprise isolating or purifying the fusion protein or isolating or purifying the protein of interest from the plant VCP. The protein of interest can be separated or purified from the plant VCP by mechanical means, such as by centrifugation or ultrafiltration, or by HPLC. The method may further comprise isolating recombinant viral particles comprising the subject polynucleotide or the subject fusion protein from the rest of the plant or plant cell.

The following examples further illustrate the present invention. These examples are intended merely to be illustrative of the present invention and are not to be construed as being limiting.

### EXAMPLES

### Biological Deposits

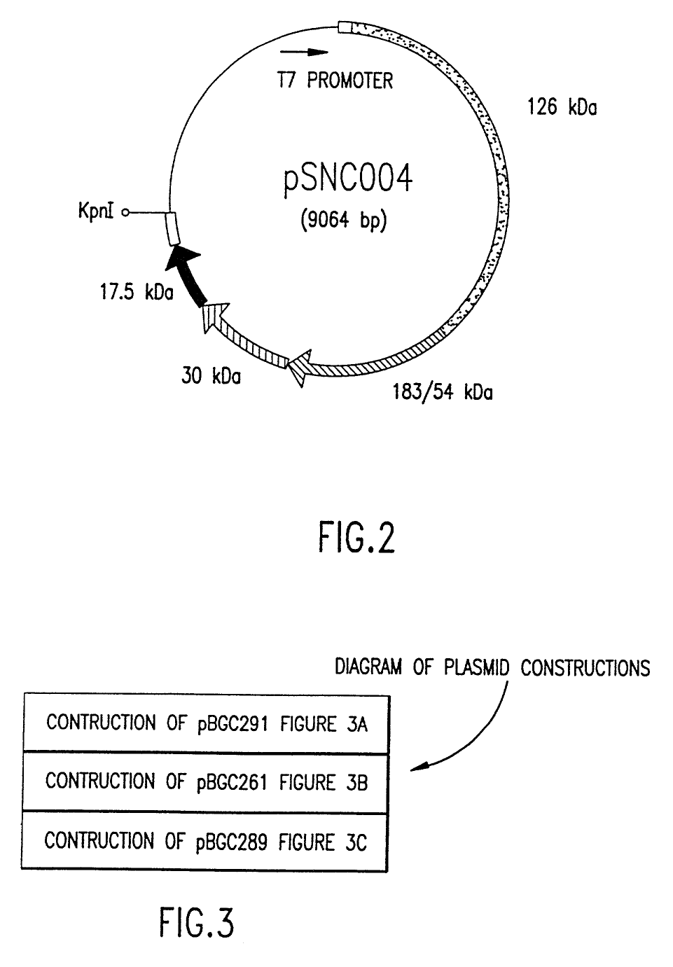

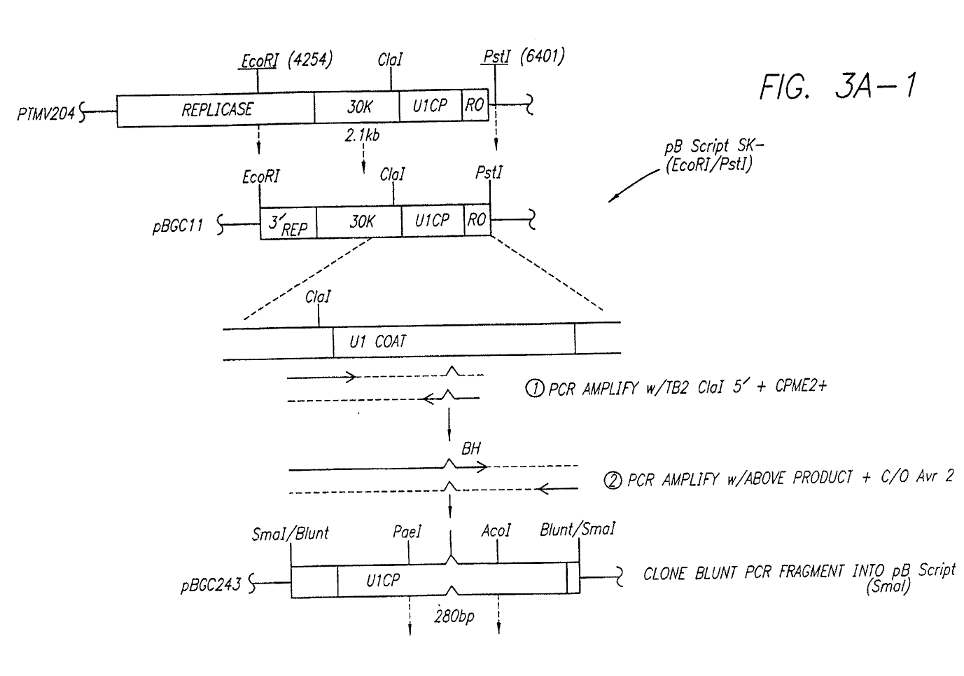

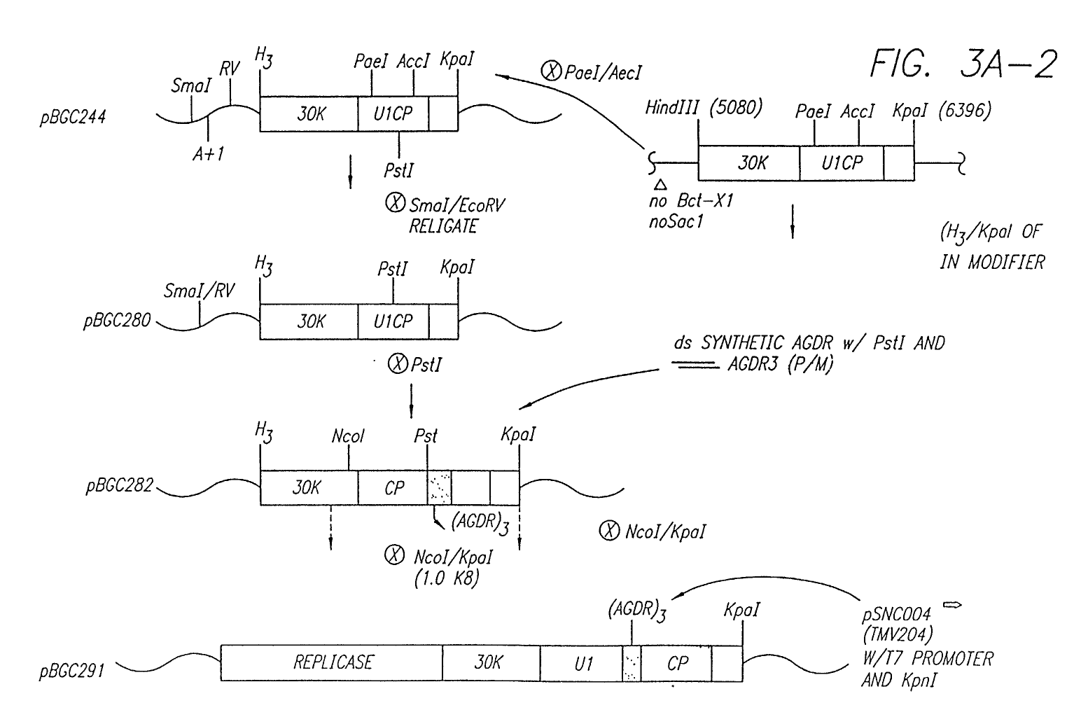

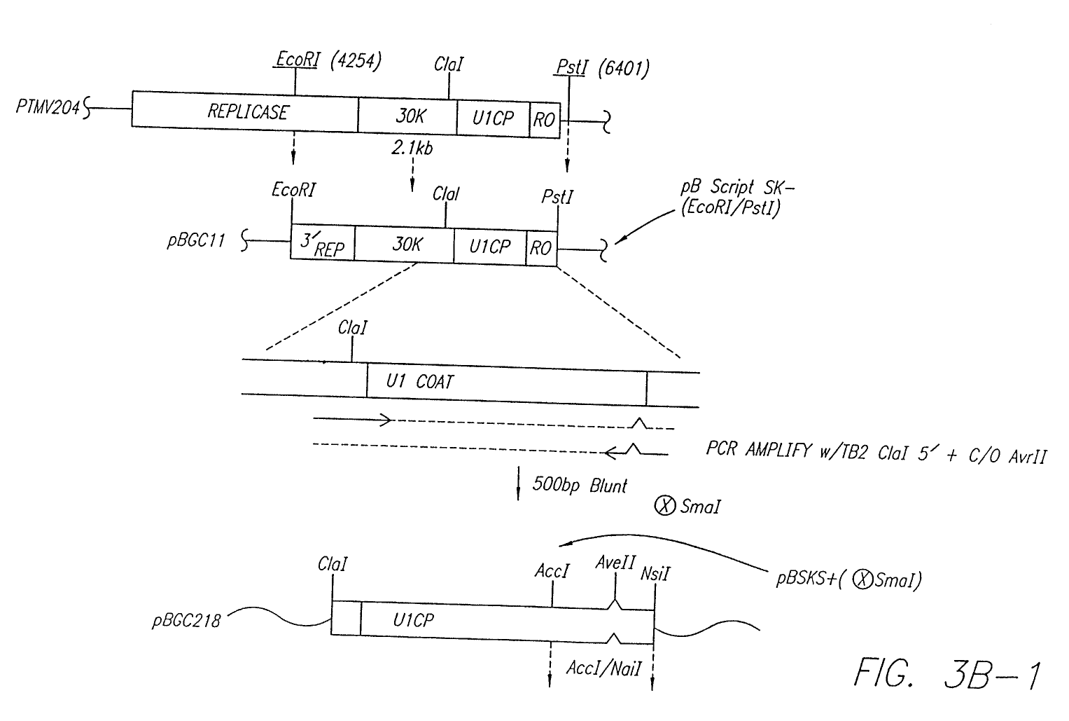

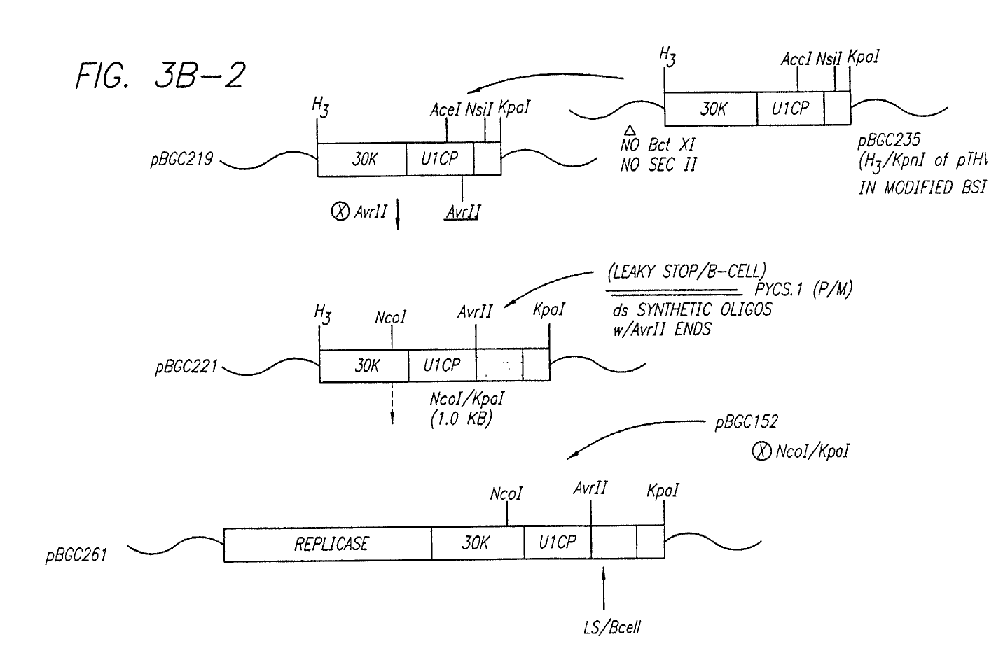

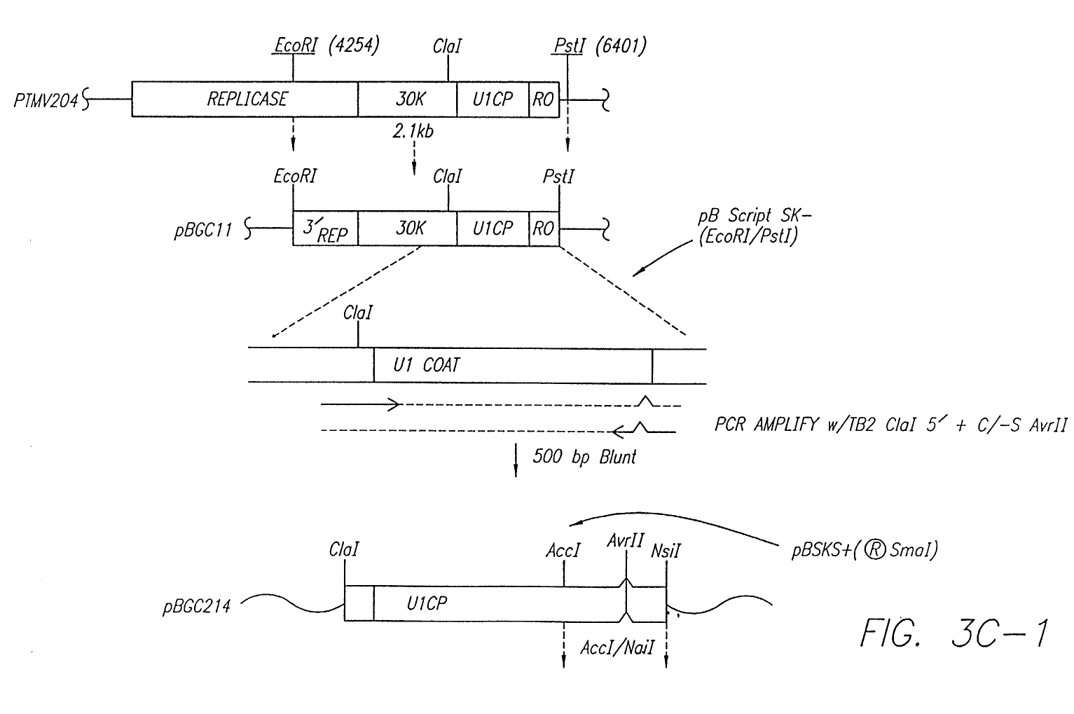

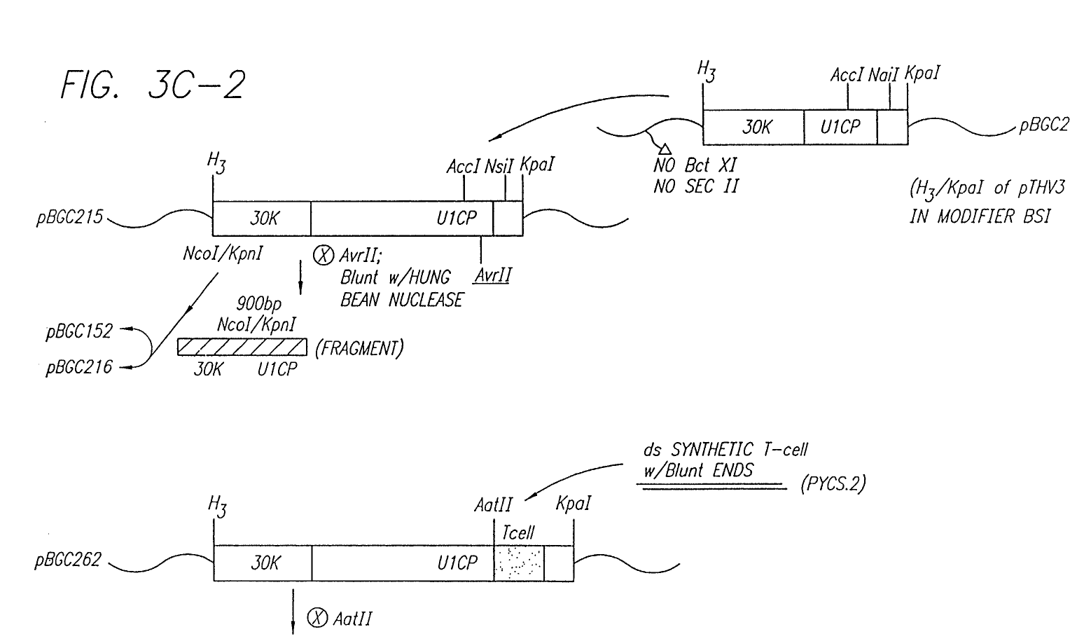

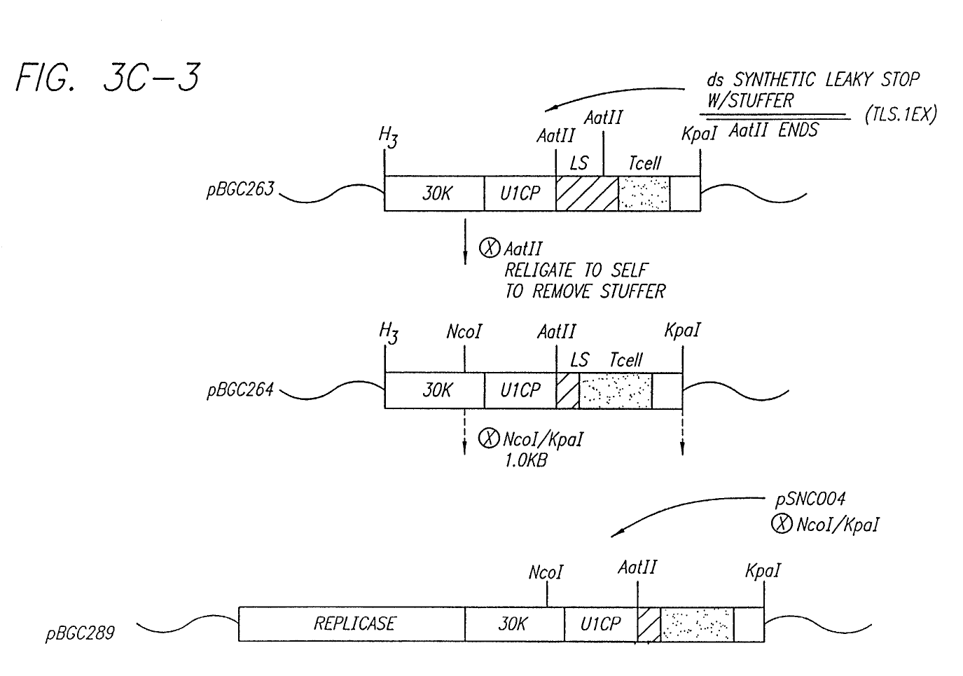

The following present examples are based on a full length insert of wild type TMV (U1 strain) cloned in the vector pUC18 with a T7 promoter sequence at the 5′-end and a KpnI site at the 3′-end (pSNC004, FIG. 2) or a similar plasmid pTMV304. Using the PCR technique and primers WD29 (SEQ ID NO: 1) and D1094 (SEQ ID NO: 2) (See Table 1 for nucleotide and amino acid sequences cited in Examples 1-4.), a 277 XmaI/HindIII amplification product was inserted with the 6140 bp XmaI/KpnI fragment from pTMV304 between the KpnI and HindIII sites of the common cloning vector pUC18 to create pSNC004. The plasmid pTMV304 is available from the American Type Culture Collection, Rockville, Md. (ATCC accession no. 45138). The genome of the wild type TMV strain can be synthesized from pTMV304 using the SP6 polymerase, or from pSNC004 using the T7 polymerase. The wild-type TMV strain can also be obtained from the American Type Culture Collection, Rockville, Md. (ATCC accession no. PV135). Plasmid pBTI 2149 and pBTI 2150 were deposited at the ATCC on Feb. 17, 2000, under the Budapest Treaty (ATCC accession nos. PTA-1403 and PTA-1404, respectively). Plasmids pJL150/198 and pJL150/199 were deposited at the ATCC (10801 University Blvd., Manassas, Va. 20110-2209) on Feb. 1, 2001 (ATCC accession nos. PTA-2984 and PTA-2983, respectively). The plasmid pBGC152, Kumagai, M., et al., (1993), is a derivative of pTMV304 and is used only as a cloning intermediate in the examples described below. The construction of each plasmid vector described in the examples below is diagrammed in FIG. 3.

TABLE 1
Nucleotide and amino acid sequences cited in
Examples 1-4.

```
SEQ ID NO: 1:
GGAATTCAAG CTTAATACGA CTCACTATAG TATTTTTACA ACAATTACC
SEQ ID NO: 2:
CCTTCATGTA AACCTCTC
SEQ ID NO: 4:
TAATCGATGA TGATTCGGAG GCTAC
SEQ ID NO: 5:
AAAGTCTCTG TCTCCTGCAG GGAACCTAAC AGTTAC
SEQ ID NO: 6:
ATTATGCATC TTGACTACCT AGGTTGCAGG ACCAGA
SEQ ID NO: 7:
GGCGATCGGG CTGGTGACCG TGCA
SEQ ID NO: 8:
CGGTCACCAG CCCGATCGCC TGCA
SEQ ID NO: 9:
ATG TCT TAC AGT ATC ACT ACT CCA TCT CAG TTC GTG TTC TTG TCA TCA
Met Ser Tyr Ser Ile Thr Thr Pro Ser Gln Phe Val Phe Leu Ser Ser
    1 5 10 15
GCG TGG GCC GAC CCA ATA GAG TTA ATT AAT TTA TGT ACT AAT GCC TTA
Ala Trp Ala Asp Pro Ile Glu Leu Ile Asn Leu Cys Thr Asn Ala Leu
                          20 25 30
GGA AAT CAG TTT CAA ACA CAA CAA GCT CGA ACT GTC GTT CAA AGA CAA
Gly Asn Gln Phe Gln Thr Gln Gln Ala Arg Thr Val Val Gln Arg Gln
                  35 40 45
TTC AGT GAG GTG TGG AAA CCT TCA CCA CAA GTA ACT GTT AGG TTC CCT
Phe Ser Glu Val Trp Lys Pro Ser Pro Gln Val Thr Val Arg Phe Pro
          50 55 60
GCA GGC GAT CGG GCT GGT GAC CGT GCA GGA GAC AGA GAC TTT AAG GTG
Ala Gly Asp Arg Ala Gly Asp Arg Ala Gly Asp Arg Asp Phe Lys Val
  65 70 75 80
TAC AGG TAC AAT GCG GTA TTA GAC CCG CTA GTC ACA GCA CTG TTA GGT
Tyr Arg Tyr Asn Ala Val Leu Asp Pro Leu Val Thr Ala Leu Leu Gly
                                  85 90 95
GCA TTC GAC ACT AGA AAT AGA ATA ATA GAA GTT GAA AAT CAG GCG AAC
Ala Phe Asp Thr Arg Asn Arg Ile Ile Glu Val Glu Asn Gln Ala Asn
                        100 105 110
CCC ACG ACT GCC GAA ACG TTA GAT GCT ACT CGT AGA GTA GAC GAC GCA
Pro Thr Thr Ala Glu Thr Leu Asp Ala Thr Arg Arg Val Asp Asp Ala
                115 120 125
ACG GTG GCC ATA AGG AGC GCG ATA AAT AAT TTA ATA GTA GAA TTG ATC
Thr Val Ala Ile Arg Ser Ala Ile Asn Asn Leu Ile Val Glu Leu Ile
        130 135 140
AGA GGA ACC GGA TCT TAT AAT CGG AGC TCT TTC GAG AGC TCT TCT GGT
Arg Gly Thr Gly Ser Tyr Asn Arg Ser Ser Phe Glu Ser Ser Ser Gly
145 150 155 160
TTG GTT TGG ACC TCT GGT CCT GCA ACT TGA SEQ ID NO:10:
Leu Val Trp Thr Ser Gly Pro Ala Thr
                                165 169
SEQ ID NO:10:
Met Ser Tyr Ser Ile Thr Thr Pro Ser Gln Phe Val Phe Leu Ser Ser
    1 5 10 15
Ala Trp Ala Asp Pro Ile Glu Leu Ile Asn Leu Cys Thr Asn Ala Leu
                          20 25 30
Gly Asn Gln Phe Gln Thr Gln Gln Ala Arg Thr Val Val Gln Arg Gln
                  35 40 45
Phe Ser Glu Val Trp Lys Pro Ser Pro Gln Val Thr Val Arg Phe Pro
          50 55 60
Ala Gly Asp Arg Ala Gly Asp Arg Ala Gly Asp Arg Asp Phe Lys Val
  65 70 75 80
Tyr Arg Tyr Asn Ala Val Leu Asp Pro Leu Val Thr Ala Leu Leu Gly
                                  85 90 95
Ala Phe Asp Thr Arg Asn Arg Ile Ile Glu Val Glu Asn Gln Ala Asn
                        100 105 110
Pro Thr Thr Ala Glu Thr Leu Asp Ala Thr Arg Arg Val Asp Asp Ala
                115 120 125
Thr Val Ala Ile Arg Ser Ala Ile Asn Asn Leu Ile Val Glu Leu Ile
        130 135 140
Arg Gly Thr Gly Ser Tyr Asn Arg Ser Ser Phe Glu Ser Ser Ser Gly
145 150 155 160
Leu Val Trp Thr Ser Gly Pro Ala Thr
                                165 169
SEQ ID NO: 12:
CTAGCAATTA CAAGGTCCAG GTGCACCTCA AGGTCCTGGA GCTCC
SEQ ID NO: 13:
CTAGGGAGCT CCAGGACCTT GAGGTGCACC TGGACCTTGT AATTG
SEQ ID NO: 15:
Met Ser Tyr Ser Ile Thr Thr Pro Ser Gln Phe Val Phe Leu Ser Ser
    1 5 10 15
Ala Trp Ala Asp Pro Ile Glu Leu Ile Asn Leu Cys Thr Asn Ala Leu
                          20 25 30
Gly Asn Gln Phe Gln Thr Gln Gln Ala Arg Thr Val Val Gln Arg Gln
                  35 40 45
Phe Ser Glu Val Trp Lys Pro Ser Pro Gln Val Thr Val Arg Phe Pro
          50 55 60
Asp Ser Asp Phe Lys Val Tyr Arg Tyr Asn Ala Val Leu Asp Pro Leu
  65 70 75 80
Val Thr Ala Leu Leu Gly Ala Phe Asp Thr Arg Asn Arg Ile Ile Gln
                                  85 90 95
Val Glu Asn Gln Ala Asn Pro Thr Thr Ala Glu Thr Leu Asp Ala Thr
                        100 105 110
Arg Arg Val Asp Asp Ala Thr Val Ala Ile Arg Ser Ala Ile Asn Asn
                115 120 125
Leu Ile Val Glu Leu Ile Arg Gly Thr Gly Ser Tyr Asn Arg Ser Ser
        130 135 140
Phe Glu Ser Ser Ser Gly Leu Val Trp Thr Ser Gly Pro Ala Thr Tyr
145 150 155 160
Gln Leu Gln Gly Pro Gly Ala Pro Gln Gly Pro Gly Ala Pro
                                165 170 174
SEQ ID NO:16:
ATG TCT TAC AGT ATC ACT ACT CCA TCT CAG TTC GTG TTC TTG TCA TCA
Met Ser Tyr Ser Ile Thr Thr Pro Ser Gln Phe Val Phe Leu Ser Ser
    1 5 10 15
GCG TGG GCC GAC CCA ATA GAG TTA ATT AAT TTA TGT ACT AAT GCC TTA
Ala Trp Ala Asp Pro Ile Glu Leu Ile Asn Leu Cys Thr Asn Ala Leu
                          20 25 30
GGA AAT CAG TTT CAA ACA CAA CAA GCT CGA ACT GTC GTT CAA AGA CAA
Gly Asn Gln Phe Gln Thr Gln Gln Ala Arg Thr Val Val Gln Arg Gln
                  35 40 45
TTC AGT GAG GTG TGG AAA CCT TCA CCA CAA GTA ACT GTT AGG TTC CCT
Phe Ser Glu Val Trp Lys Pro Ser Pro Gln Val Thr Val Arg Phe Pro
          50 55 60
GAC AGT GAC TTT AAG GTG TAC AGG TAC AAT GCG CTA TTA GAC CCG CTA
Asp Ser Asp Phe Lys Val Tyr Arg Tyr Asn Ala Val Leu Asp Pro Leu
  65 70 75 80
GTC ACA GCA CTG TTA GGT GCA TTC GAC ACT AGA AAT AGA ATA ATA GAA
Val Thr Ala Leu Leu Gly Ala Phe Asp Thr Arg Asn Arg Ile Ile Glu
                                  85 90 95
GTT GAA AAT CAG GCG AAC CCC ACG ACT GCC GAA ACG TTA GAT GCT ACT
Val Glu Asn Gln Ala Asn Pro Thr Thr Ala Glu Thr Leu Asp Ala Thr
                        100 105 110
CGT AGA GTA GAC GAC GCA ACG GTG GCC ATA AGG AGC GCG ATA AAT AAT
Arg Arg Val Asp Asp Ala Thr Val Ala Ile Arg Ser Ala Ile Asn Asn
                115 120 125
TTA ATA GTA GAA TTG ATC AGA GGA ACC GGA TCT TAT AAT CGG AGC TCT
Leu Ile Val Glu Leu Ile Arg Gly Thr Gly Ser Tyr Asn Arg Ser Ser
        130 135 140
TTC GAG AGC TCT TCT GGT TTG GTT TGG ACC TCT GGT CCT GCA ACC TAG
Phe Glu Ser Ser Ser Gly Leu Val Trp Thr Ser Gly Pro Ala Thr
145 150 155 159
SEQ ID NO:17:
Met Ser Tyr Ser Ile Thr Thr Pro Ser Gln Phe Val Phe Leu Ser Ser
    1 5 10 15
Ala Trp Ala Asp Pro Ile Glu Leu Ile Asn Leu Cys Thr Asn Ala Leu
                          20 25 30
Gly Asn Gln Phe Gln Thr Gln Gln Ala Arg Thr Val Val Gln Arg Gln
                  35 40 45
Phe Ser Glu Val Trp Lys Pro Ser Pro Gln Val Thr Val Arg Phe Pro
          50 55 60
Asp Ser Asp Phe Lys Val Tyr Arg Tyr Asn Ala Val Leu Asp Pro Leu
  65 70 75 80
Val Thr Ala Leu Leu Gly Ala Phe Asp Thr Arg Asn Arg Ile Ile Glu
                                  85 90 95
Val Glu Asn Gln Ala Asn Pro Thr Thr Ala Glu Thr Leu Asp Ala Thr
                        100 105 110
Arg Arg Val Asp Asp Ala Thr Val Ala Ile Arg Ser Ala Ile Asn Asn
                115 120 125
Leu Ile Val Glu Leu Ile Arg Gly Thr Gly Ser Tyr Asn Arg Ser Ser
        130 135 140
Phe Glu Ser Ser Ser Gly Leu Val Trp Thr Ser Gly Pro Ala Thr
145 150 155 159
SEQ ID NO: 19:
ATTATGCATC TTGACTACCT AGGTCCAAAC CAAAC
SEQ ID NO: 20:
GTCATATGTT CCATCTGCAG AGCAGATCTT GGAATTCGTT AAGCAAATCT
CGAGTCAGTA ACTATA
SEQ ID NO: 21:
TATAGTTACT GACTCGAGAT TTGCTTAACG AATTCCAAGA TCTGCTCTGC
AGATGGAACA TATGAC
SEQ ID NO: 22:
CGACCTAGGT GATGACGTCA TAGCAATTAA CGT
SEQ ID NO: 23:
TAATTGCTAT GACGTCATCA CCTAGGTCGA CGT
```

### Example 1

### Propagation and Purification of the U1 Strain of TMV

The TMVCP fusion vectors described in the following examples are based on the U1 or wild-type TMV strain and are therefore compared to the parental virus as a control. Nicotiana tabacum cv Xanthi (hereafter referred to as tobacco) was grown 4-6 weeks after germination, and two 4-8 cm expanded leaves were inoculated with a solution of 50 μg/ml TMV U1 by pipetting 100 μl onto carborundum dusted leaves and lightly abrading the surface with a gloved hand. Six tobacco plants were grown for 27 days post inoculation accumulating 177 g fresh weight of harvested leaf biomass not including the two lower inoculated leaves. Purified TMV U1 Sample ID No. TMV204.B4 was recovered (745 mg) at a yield of 4.2 mg of virion per g of fresh weight by two cycles of differential centrifugation and precipitation with PEG according to the method of Gooding, et al. (1967). Tobacco plants infected with TMV U1 accumulated greater than 230 micromoles of CP per kg of leaf tissue.

### Example 2

### Production of a Malarial B-Cell Epitope Genetically Fused to the Surface Loop Region of the TMVCP

The monoclonal antibody NVS3 was made by immunizing a mouse with irradiated P. vivax sporozoites. NVS3 mAb passively transferred to monkeys provided protective immunity to sporozoite infection with this human parasite. Using the technique of epitope-scanning with synthetic peptides, the exact amino acid sequence present on the P. vivax sporozoite surface and recognized by NVS3 was defined as AGDR (SEQ ID NO. 3). The epitope AGDR is contained within a repeating unit of the circumsporozoite (CS) protein (Charoenvit et al., 1991a), the major immunodominant protein coating the sporozoite. Construction of a genetically modified tobamovirus designed to carry this malarial B-cell epitope fused to the surface of virus particles is set forth herein.

Construction of Plasmid pBGC291

The 2.1 kb EcoRI-PstI fragment from pTMV204 described in Dawson, W., et al. (1986) was cloned into pBstSK− (Stratagene Cloning Systems) to form pBGC11. A 0.27 kb fragment of pBGC11 was PCR amplified using the 5′ primer TB2ClaI5′ (SEQ ID NO: 4) and the 3′ primer CP.ME2+ (SEQ ID NO: 5) (see Table 1). The 0.27 kb amplified product was used as the 5′ primer and C/0AvrII (SEQ ID NO: 6) (see Table 1) was the 3′ primer for PCR amplification. The amplified product was cloned into the SmaI site of pBstKS+ (Stratagene Cloning Systems) to form pBGC243. To eliminate the BstXI and SacII sites from the polylinker, pBGC234 was formed by digesting pBstKS+ (Stratagene Cloning Systems) with BstXI followed by treatment with T4 DNA Polymerase and self-ligation. The 1.3 kb HindIII-KpnI fragment of pBGC304 was cloned into pBGC234 to form pBGC235. pBGC304 is also named pTMV304 (ATCC deposit 45138). The 0.3 kb PacI-AccI fragment of pBGC243 was cloned into pBGC235 to form pBGC244. The 0.02 kb polylinker fragment of pBGC243 (SmaI-EcoRV) was removed to form pBGC280. A 0.02 kb synthetic PstI fragment encoding the P. vivax AGDR repeat was formed by annealing AGDR3p (SEQ ID NO: 7) with AGDR3m (SEQ ID NO: 8) (see Table 1) and the resulting double stranded fragment was cloned into pBGC280 to form pBGC282. The 1.0 kb NcoI-KpnI fragment of pBGC282 was cloned into pSNC004 to form pBGC291. The CP sequence of the virus TMV291 produced by transcription of plasmid pBGC291 in vitro is listed in (SEQ ID NO: 9) (the amino acid sequence alone is listed in SEQ ID NO: 10) (see Table 1). The epitope (AGDR)3 is calculated to be approximately 6.2% of the weight of the virion.

Propagation and Purification of the Epitope Expression Vector

Infectious transcripts were synthesized from KpnI-linearized pBGC291 using T7 RNA polymerase and cap (7mGpppG) according to the manufacturer (New England Biolabs). An increased quantity of recombinant virus was obtained by passaging and purifying Sample ID No. TMV291.1B1 as described in Example 1. Twenty tobacco plants were grown for 29 days post inoculation, accumulating 1060 g fresh weight of harvested leaf biomass not including the two lower inoculated leaves. Purified Sample ID TMV291.1B2 was recovered (474 mg) at a yield of 0.4 mg virion per g of fresh weight. Therefore, 25 μg of 12-mer peptide was obtained per g of fresh weight extracted. Tobacco plants infected with TMV291 accumulated greater than 21 micromoles of peptide per kg of leaf tissue.

Product Analysis

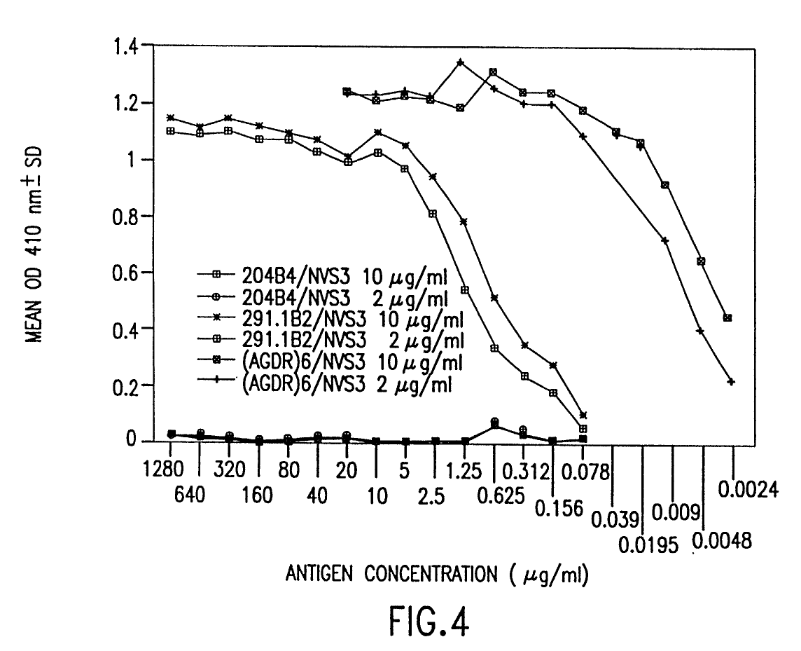

The conformation of the epitope AGDR contained in the virus TMV291 is specifically recognized by the monoclonal antibody NVS3 in ELISA assays (FIG. 4). By Western blot analysis, NVS3 cross-reacted only with the TMV291 cp fusion at 18.6 kD and did not cross-react with the wild type or CP fusion present in TMV261. The genomic sequence of the epitope coding region was confirmed by directly sequencing viral RNA extracted from Sample ID No. TMV291.1B2.

### Example 3

### Production of a Malarial B-Cell Epitope Genetically Fused to the C Terminus of the TMVCP

Significant progress has been made in designing effective subunit vaccines using rodent models of malarial disease caused by nonhuman pathogens such as P. yoelii or P. berghei. The monoclonal antibody NYS1 recognizes the repeating epitope QGPGAP (SEQ ID NO: 11), present on the CS protein of P. yoelii, and provides a very high level of immunity to sporozoite challenge when passively transferred to mice (Charoenvit, et al. 1991b). Construction of a genetically modified tobamovirus designed to carry this malarial B-cell epitope fused to the surface of virus particles is set forth herein.

Construction of Plasmid pBGC261

A 0.5 kb fragment of pBGC11, was PCR amplified using the 5′ primer TB2ClaI5′ (SEQ ID NO: 4) and the 3′ primer C/0AvrII (SEQ ID NO: 6). The amplified product was cloned into the SmaI site of pBstKS+ (Stratagene Cloning Systems) to form pBGC218. pBGC219 was formed by cloning the 0.15 kb AccI-NsiI fragment of pBGC218 into pBGC235. A 0.05 kb synthetic AvrII fragment was formed by annealing PYCS.1p (SEQ ID NO: 12) with PYCS.1m (SEQ ID NO: 13) (see Table 1) and the resulting double stranded fragment, encoding the leaky-stop signal and the P. yoelii B-cell malarial epitope, was cloned into the AvrII site of pBGC219 to form pBGC221. The 1.0 kb NcoI-KpnI fragment of pBGC221 was cloned into pBGC152 to form pBGC261. The virus TMV261, produced by transcription of plasmid pBGC261 in vitro, contains a leaky stop signal at the C terminus of the CP gene and is therefore predicted to synthesize wild-type and recombinant coat proteins at a ratio of 20:1. The recombinant TMVCP fusion synthesized by TMV261 is listed in (SEQ ID NO: 14) (the amino acid sequence alone is listed in SEQ ID NO: 15) (see Table 1) with the stop codon decoded as the amino acid Y (amino acid residue 160). The wild-type sequence, synthesized by the same virus, is listed in (SEQ ID NO: 16) (the amino acid sequence alone is listed in SEQ ID NO: 17) (see Table 1). The epitope (QGPGAP)2 is calculated to be present at 0.3% of the weight of the virion.

Propagation and Purification of the Epitope Expression Vector

Infectious transcripts were synthesized from KpnI-linearized pBGC261 using SP6 RNA polymerase and cap (7mGpppG) according to the manufacturer (Gibco/BRL Life Technologies). An increased quantity of recombinant virus was obtained by passaging and purifying Sample ID No. TMV261.B1b as described in Example 1. Six tobacco plants were grown for 27 days post inoculation, accumulating 205 g fresh weight of harvested leaf biomass not including the two lower inoculated leaves. Purified Sample ID No. TMV261.1B2 was recovered (252 mg) at a yield of 1.2 mg virion per g of fresh weight. Therefore, 4 μg of 12-mer peptide was obtained per g of fresh weight extracted. Tobacco plants infected with TMV261 accumulated greater than 3.9 micromoles of peptide per kg of leaf tissue.

Product Analysis

The content of the epitope QGPGAP in the virus TMV261 was determined by ELISA with monoclonal antibody NYS1 (FIG. 5). From the titration curve, 50 μg/ml of TMV261 gave the same O.D. reading (1.0) as 0.2 ug/ml of (QGPGAP)2. The measured value of approximately 0.4% of the weight of the virion as epitope is in good agreement with the calculated value of 0.3%. By Western blot analysis, NYS1 cross-reacted only with the TMV261 CP fusion at 19 kD and did not cross-react with the wild-type CP or CP fusion present in TMV291. The genomic sequence of the epitope coding region was confirmed by directly sequencing viral RNA extracted from Sample ID. No. TMV261.1B2.

### Example 4

### Production of a Malarial CTL Epitope Genetically Fused to the C Terminus of the TMVCP

Malarial immunity induced in mice by irradiated sporozoites of P. yoelii is also dependent on CD8+ T lymphocytes. Clone B is one cytotoxic T lymphocyte (CTL) cell clone shown to recognize an epitope present in both the P. yoelii and P. berghei CS proteins. Clone B recognizes the following amino acid sequence; SYVPSAEQILEFVKQISSQ (SEQ ID NO: 18) and when adoptively transferred to mice protects against infection from both species of malaria sporozoites (Weiss, et al., 1992). Construction of a genetically modified tobamovirus designed to carry this malarial CTL epitope fused to the surface of virus particles is set forth herein.

Construction of Plasmid pBGC289

A 0.5 kb fragment of pBGC11 was PCR amplified using the 5′ primer TB2ClaI5′ (SEQ ID NO: 4) and the 3′ primer C/−5AvrII (SEQ ID NO: 19) (see Table 1). The amplified product was cloned into the SmaI site of pBstKS+ (Stratagene Cloning Systems) to form pBGC214. pBGC215 was formed by cloning the 0.15 kb AccI-NsiI fragment of pBGC214 into pBGC235. The 0.9 kb NcoI-KpnI fragment from pBGC215 was cloned into pBGC152 to form pBGC216. A 0.07 kb synthetic fragment was formed by annealing PYCS.2p (SEQ ID NO: 20) with PYCS.2m (SEQ ID NO: 21) (see Table 1) and the resulting double stranded fragment, encoding the P. yoelii CTL malarial epitope, was cloned into the AvrII site of pBGC215 made blunt ended by treatment with mung bean nuclease and creating a unique AatII site, to form pBGC262. A 0.03 kb synthetic AatII fragment was formed by annealing TLS.1EXP (SEQ ID NO: 22) with TLS.1EXM (SEQ ID NO: 23) (see Table 1) and the resulting double stranded fragment, encoding the leaky-stop sequence and a stuffer sequence used to facilitate cloning, was cloned into AatII digested pBGC262 to form pBGC263. pBGC262 was digested with AatII and ligated to itself removing the 0.02 kb stuffer fragment to form pBGC264. The 1.0 kb NcoI-KpnI fragment of pBGC264 was cloned into pSNC004 to form pBGC289. The virus TMV289 produced by transcription of plasmid pBGC289 in vitro, contains a leaky stop signal resulting in the removal of four amino acids from the C terminus of the wild-type TMV CP gene and is therefore predicted to synthesize a truncated CP and a CP with a CTL epitope fused at the C terminus at a ratio of 20:1. The recombinant TMVCP/CTL epitope fusion present in TMV289 (encoded in the nucleotide sequence listed in SEQ ID NO: 24) is listed in SEQ ID NO: 25 (see Table 1) with the stop codon decoded as the amino acid Y (amino acid residue 156). The wild-type sequence minus four amino acids from the C terminus is listed in SEQ ID NO: 26 (the amino acid sequence alone is listed in SEQ ID NO: 27) (see Table 1). The amino acid sequence of the CP of virus TMV216 produced by transcription of the plasmid pBGC216 in vitro, is also truncated by four amino acids. The epitope SYVPSAEQILEFVKQISSQ (SEQ ID NO: 18) is calculated to be present at approximately 0.5% of the weight of the virion using the same assumptions confirmed by quantitative ELISA analysis of the readthrough properties of TMV261 in Example 3.

Propagation and Purification of the Epitope Expression Vector

Infectious transcripts were synthesized from KpnI-linearized pBGC289 using T7 RNA polymerase and cap (7mGpppG) according to the manufacturer (New England Biolabs). An increased quantity of recombinant virus was obtained by passaging Sample ID No. TMV289.11B1a as described in Example 1. Fifteen tobacco plants were grown for 33 days post inoculation accumulating 595 g fresh weight of harvested leaf biomass not including the two lower inoculated leaves. Purified Sample ID. No. TMV289.11B2 was recovered (383 mg) at a yield of 0.6 mg virion per g of fresh weight. Therefore, 3 μg of 19-mer peptide was obtained per g of fresh weight extracted. Tobacco plants infected with TMV289 accumulated greater than 1.4 micromoles of peptide per kg of leaf tissue.

Product Analysis

Partial confirmation of the sequence of the epitope coding region of TMV289 was obtained by restriction digestion analysis of PCR amplified cDNA using viral RNA isolated from Sample ID. No. TMV289.11B2. The presence of proteins in TMV289 with the predicted mobility of the CP fusion at 20 kD and the truncated CP at 17.1 kDa was confirmed by denaturing PAGE.

### Example 5

### Construction of pJL 60.3

To facilitate cloning of TMV U1 CP fusions into an infectious TMV U1 cDNA backbone, the vector pJL 60.3 was constructed. The plasmid pJL 60.3 contains a full length infectious clone of TMV U1 with a small multiple cloning site polylinker: TAAATATTCTTAAGCCAGTAGTATGGGATATCCAGTGGTATGGGATCCTAC AGTATC (SEQ ID NO: 28) containing two BstXI sites, CCAGTAGTATGG (SEQ ID NO: 29) and CCAGTGGTATGG (SEQ ID NO: 30), separated by a unique EcoRV site (underlined), between the stop codon of the 30K protein gene and the start codon of the U1 CP. To construct pJL 60.3, a 0.7 kb DNA fragment comprising the TMV U1 CP and 3′ UTS was PCR amplified from pBTI 801 using the following primers:

kinased 5′ primer JAL 72

TGGGATATCCAGTGGTATGGGATCCTACAGTATACACTACTCCATCTCAG (SEQ ID NO:31) and

3′ primer JON 56

CGCGTACCTGGGCCCCTACCGGGGGTAACG (SEQ ID NO: 32).

pBTI 801 contains a full length infectious clone TMV U1, under the control of the T7 promoter sequence, in a pUC based plasmid. A KpnI restriction enzyme site lies at the 3′ end of the viral cDNA, immediately followed by a self-processing ribozyme sequence from satellite tobacco ringspot virus RNA. The presence of this self-processing ribozyme downstream of the TMV 3′ end allows for the transcription of the TMV cDNA without prior linearization of the plasmid template DNA (e.g., with KpnI).

A 0.3 kb fragment of pBTI 801 was then PCR amplified using the following primers:

5′ primer JON 52 (TMV U1 nts 5456-5482):

GGCCCATGGAACTTACAGAAGAAGTCG (SEQ ID NO: 33) and

kinased 3′ primer JAL 73

CTGGATATCCCATACTACTGGCTTAAGAATATTTAAAACGAATCCGATTCG GCGACA (SEQ ID NO: 34).

The 0.7 kb PCR product, containing the EcoRV and BstXI site CCAGTGGTATGG (SEQ ID NO: 30) upstream of the U1 CP ORF and 3′ UTS, was then ligated to the 0.3 bp PCR products (which contained the 3′ end of the TMV 30K protein gene and the BstXI site CCAGTAGTATGG (SEQ ID NO: 29) downstream of the 30K protein stop codon. The product of this ligation reaction was then used in a PCR with 5′ primer JON 52 (shown above) and 3′ primer JON56 (shown above) to generate a 1 kb PCR product. That product was digested with PacI and NcoI, and the digested DNA was electrophoresed on an agarose gel. The NcoI site is contained within the primer sequence of JON 52, and the PacI site is a unique restriction site in the TMV U1 CP gene sequence. The 0.4 kb PacI-NcoI fragment was then isolated from an agarose gel and ligated into a PacI-NcoI digested 8.8 kb fragment of pBTI 801 to generate pJL 60.3. The relevant feature of pJL 60.3 for the construction of pBTI 2149 and pBTI 2150 is the existence of the BstXI site CCAGTAGTATGG (SEQ ID NO: 29) between the TMV 30K stop codon and the CP start codon.

### Example 6

### Construction of Plasmid pBTI 2149

A 0.7 kb DNA fragment comprising the TMV U1 coat protein (CP) and 3′ UTS was PCR amplified from p BTI 801 using the following primers:

5′ primer JAL 149

CCTGGGCCAGTAGTATGGGTTCAGATGGTGCTGTACAACCAGATGGAGGT CAACCAGCTGTATCTTACAGTATCACTACTCCATCTCAGTT (SEQ ID NO: 35) and

3′ primer JON 56 p1 (shown above).

JAL 149 contains the BstXI restriction enzyme site (underlined) for cloning purposes and the coding sequence for the parovirus epitope MGSDGAVQPDGGQPAV (SEQ ID NO: 36) and TMV U1 nts 5715-5743. The amplified product comprising the parvovirus epitope fused to the U1 CP gene was digested with KpnI and BstXI and ligated into the 8.4 kb KpnI-BstXI fragment of pJL 60.3 to generate pBTI 2149. Plasmid vectors pBTI 2149 encodes the recombinant virus having a fusion protein of MGSDGAVQPDGGQPAV (SEQ ID NO: 36) fused to the N-terminus of the coat protein.

### Example 7

### Construction of Plasmid pBTI 2150

A 0.7 kb DNA fragment comprising the TMV U1 CP and 3′ UTS was PCR amplified from p801 (basically pTMV 204) using the following primers:

5′ primer JAL 150

CCTGGGCCAGTAGTATGGGTTCAGATGGTGCTGTACAACCAGATGGAGGT CAACCAGCTGTATCTTACAGTATCACTACTCCATCTCAGTT (SEQ ID NO: 37) and

3′ primer JON 56

(shown above)

The “forward” primer JAL 150 contains a BstXI restriction enzyme site (underlined) for cloning purposes, the coding sequence for the parovirus epitope MGQPDGGQPAVRNERAT (SEQ ID NO: 38) and TMV U1 nts 5718-5743. The amplified product comprising the parvovirus epitope fused to the U1 CP gene was digested with KpnI and BstXI and ligated into the 8.4 kb KpnI-BstXI fragment of pJL 60.3 to generate pBTI 2150. Plasmid vectors pBTI 2150 encodes the recombinant virus having a fusion protein of MGQPDGGQPAVRNERAT (SEQ ID NO: 38) fused to the N-terminus of the CP.

### Example 8

### Production of Virus TMV 149

The virus TMV 149 was produced by transcription of plasmid pBTI 2149. Infectious transcripts were synthesized from transcription reactions with T7 RNA polymerase in the presence of cap analog (7mGpppG) (New England Biolabs) according to the manufacturer's instructions. Transcripts were used to inoculate N. benthamiana and N. tabacum leaves which had been lightly dusted with carborundum (silicon carbide 400 mesh, Aldrich).

### Example 9

### Production of Virus TMV 150

The virus TMV 150 was produced by transcription of plasmid pBTI 2150. Infectious transcripts were synthesized from transcription reactions with T7 RNA polymerase in the presence of cap analog (7mGpppG) (New England Biolabs) according to the manufacturer's instructions. Transcripts were used to inoculate N. benthamiana and N. tabacum leaves that had been lightly dusted with carborundum (silicon carbide 400 mesh, Aldrich).

### Example 10

### Extraction and Purification of TMV CP Fusion Virions

The two TMV coat fusion constructs, TMV149 and TMV150, were expressed in and extracted from N. benthamiana and/or N. tabacum using a pH-heat or PEI extraction method as described below, and in Table 1. Virus preparations were characterized using MALDI-TOF (Example 11; see Table 3). Based upon the product masses determined by MALDI and PAGE analysis, a proteolytic degradation profile was determined for each construct for any given host plant or extraction method used to produce the coat fusion product (See Tables 3 and 4).

pH-Heat Extraction

N. benthamiana or N. tabacum cv MD609, produced in a growth rooms, were inoculated with TMV derivatives containing parvovirus epitopes fused to the N-terminus of the coat protein (TMV149 and TMV150 fusions). Plants were harvested 2.5-5 weeks post inoculation after systemic spread of the virus Leaf and stalk tissue (150 g) was macerated in a 1 L Waring blender for 2.0 min. at the high setting with 300 ml of chilled, 0.04% Na₂S₂O₅. The macerated material was strained through four layers of cheesecloth to remove fibrous material. The resultant “green juice” was adjusted to a pH of 5.0 with H₃PO₄. The pH adjusted green juice was heated to 47° C. and held at this temperature for 5 min. and then cooled to 15° C. The heat-treated green juice was centrifuged at 6,000×G for 3 min. resulting in two fractions, supernatant 1 and pellet 1. The pellet 1 fraction was resuspended in distilled water using a volume of water equivalent to 1/z of the initial green juice volume. The resuspended pellet 1 was adjusted to a pH of 7.5 with NaOH and centrifuged at 6,000×G for 3 min. resulting in two fractions, supernatant 2 and pellet 2. Virus was precipitated from both supernatant fractions 1 and 2 by the addition of PEG 6,000 and NaCl (4% by volume). After incubation at 4° C. (1 hour), precipitated virus was recovered by centrifugation at 10,000×G for 10 min. The virus pellet was resuspended in 10 mM NaKPO₄ buffer, pH 7.2 and clarified by centrifugation at 10,000×G for 3 min. The clarified virus preparation was precipitated a second time by the addition of PEG 6,000 and NaCl (4% by volume). Precipitated virus was recovered by centrifugation as described above. Virus yields are shown in Table 2.

PEI Extraction

N. benthamiana or N. tabacum cv MD609, produced in a growth rooms, were inoculated with TMV derivatives containing parvovirus epitopes fused to the N-terminus of the CP (TMV149 and TMV150 fusions). Plants were harvested 2.5-5 weeks post inoculation after systemic spread of the virus. Leaf and stalk tissue (150 g) was macerated in a 1 L Waring blender for 2.0 min. at the high setting with 300 ml of chilled, 50 mM Tris, pH 7.5, 2 mM EDTA and 0.1% β-mercaptoethanol. The macerated material was strained through four layers of cheesecloth to remove fibrous material. The resultant “green juice” was adjusted to 0.1% PEI (Sigma, St. Louis, Mo.) by the addition of a 10% PEI W/V stock solution. The PEI treated green juice was stirred for 30 min., (4° C.) and then centrifuged at 3,000×G for 5 min. resulting in two fractions, supernatant 1 and pellet 1. The pellet 1 fraction was resuspended in distilled water using a volume of water equivalent to ½ of the initial green juice volume. The resuspended pellet 1 was adjusted to a pH of 7.5 with NaOH and centrifuged at 6,000×G for 3 min. resulting in two fractions, supernatant 2 and pellet 2. Virus was precipitated from both supernatant fractions 1 and 2 by the addition of PEG 6,000 and NaCl (4% by volume). After incubation at 4° C. (1 hour), precipitated virus was recovered by centrifugation at 10,000×G for 10 min. The virus pellet was resuspended in 10 mM NaKPO₄ buffer, pH 7.2 and clarified by centrifugation at 10,000×G for 3 min. The clarified virus preparation was precipitated a second time by the addition of PEG 6,000 and NaCl (4% by volume). Precipitated virus was recovered by centrifugation as described above. Virus yields are shown in Table 2.

The yield of epitope specific virus particles is dependent upon the species of plant used as the virus host and method of extraction. TMV149 yielded the highest quantity of virus when produced in N. benthamiana and extracted using the pH-heat method. In addition, the TMV149 particles partitioned primarily into supernatant 1. Negligible yields of TMV149 were observed when the PEI method was employed. TMV150 yielded the highest quantity of virus when produced in N. benthamiana and extracted using the PEI method. TMV150 partitioned into both supernatant 1 and 2 (60% and 40%, respectively) when extracted by the pH-heat method.

TABLE 2
Virus Yield

| Vector | Host Plant | Extraction Method | Virus Yield* |
|---|---|---|---|
| TMV149 | N. benthamiana | pH-Heat, Supernatant 1 | 0.3929 |
| TMV149 | N. benthamiana | pH-Heat, Supernatant 2 | 0.0396 |
| TMV149 | N. benthamiana | PEI, Supernatant 1 | 0.0005 |
| TMV149 | N. benthamiana | PEI, Supernatant 2 | — |
| TMV149 | N. tabacum | pH-Heat, Supernatant 1 | 0.0488 |
| TMV149 | N. tabacum | pH-Heat, Supernatant 2 | 0.0376 |
| TMV149 | N. tabacum | PEI, Supernatant 1 | — |
| TMV149 | N. tabacum | PEI, Supernatant 2 | — |
| TMV150 | N. benthamiana | pH-Heat, Supernatant 1 | 1.2274 |
| TMV150 | N. benthamiana | PEI, Supernatant 2 | 0.8860 |
| TMV150 | N. benthamiana | PEI, Supernatant 1 | 1.5369 |
| TMV150 | N. tabacum | PEI, Supernatant 2 | — |
| TMV150 | N. tabacum | pH-Heat, Supernatant 1 | 0.321 |
| TMV150 | N. tabacum | PEI, Supernatant 1 | 0.0368 |
| TMV150 | N. tabacum | PEI, Supernatant 2 | 0.0001 |

*Virus yields are expressed as mg virus per g fresh weight, plant tissue extracted and were determined spectrophotometrically (absorbance at A260). All values were derived from the initial PEG precipitates.

### Example 11

### Analysis of CP Fusions by MALDI

PEG precipitated, resuspended virus preparations were diluted in 50% acetonitrile and further diluted 1:1 with sinapinic acid (Aldrich, Milwaukee, Wis.). The sinapinic acid was prepared at a concentration of 10 mg/ml in 0.1% aqueous triflouroacetic acid/acetonitrile (70/30 by volume). The sinapinic acid treated sample (1.0 μl) was applied to a stainless steel MALDI plate surface and allowed to air dry at room temperature. MALDI-TOF mass spectra were obtained with a PerSeptive Biosystems DE-PRO (Houston, Tex.) operated in the linear mode. A pulsed laser operating at 337 rim was used in the delayed extraction mode for ionization. An acceleration voltage of 25 kV with a 90% grid voltage and a 0.1% guide wire voltage was used. Approximately 100 scans were acquired and averaged over the mass range 2-156 kDa with a low mass gate of 2 kDa. Ion source and mirror pressures were approximately 1.2×10⁻⁷ and 1.6×10⁻⁷ Torr, respectively. All spectra were mass calibrated with a single-point fit using horse apomyoglobin (16,952 Da).

The results presented in Tables 3 and 4 indicate effects of host species, extraction method and extraction timing on the proteolysis of N-terminal TMV CP fusions. In all cases, the terminal Met residue is removed from all fusions, as is the case with native CP. The N-terminal glycine residue is removed from 40-60% of the TMV149 fusions. Extractions (H-heat) performed on TMV149 and 150 produced in 17 day post inoculated N. tabacum, resulted in the most complex and greatest degree of proteolytic activity. The differences in proteolytic degradation may reflect both qualitative and quantitative differences in proteases present in different plant species or at different plant development periods. The PEI extraction of TMV150 proved to be protective, resulting in negligible degradation relative to the pH-heat extraction (N. tabacum host).

TABLE 3
Product Mass Characterization

| | Days Post | Extraction Method and | Product Mass (MALDI) |
|---|---|---|---|
| Plant Host/Vector | Inoculation | Fraction | Daltons*,** |
| N. benthamiana/ | 17 | pH-Heat Supernatant 1 | 18,822 (50%); 18,766 (50%)** |
| TMV149 | | | |
| N. tabacum/ | 17 | pH-Heat Supernatant 1 | 18,823 (40%); 18,762 (40%): |
| TMV149 | | | 18,564 (<2%); 18,509 (<2%); |
| | | | 18,442 (2%); 18,329 (<2%); |
| | | | 17,993 (10%); 17,935 (2%) |
| N. tabacum/ | 35 | pH-Heat, Supernatant 1 | 18,812 (60%); 18,752 (40%) |
| TMV149 | | | |
| N. benthamiana/ | 17 | pH-Heat, Supernatant 1 | 19,025 (>95%); 17,964 (<5%) |
| TMV150 | | | |
| N. benthamiana/ | 17 | PEI, Supernatant 1 | 19,029 (>95%); 17,980 (<5%) |
| TMV150 | | | |
| N. tabacum/ | 17 | pH-Heat, Supernatant 1 | 19,020 (60%); 17,956 (40%)** |
| TMV150 | | | |
| N. tabacum/ | 35 | pH-Heat, Supernatant 1 | 19,020 (80%); 17,956 (20%) |
| TMV150 | | | |
| N. tabacum/ | 17 | PEI, Supernatant 1 | 19,021 (>95%); 17,957 (<5%) |
| TMV150 | | | |

*The number in parentheses is the approximate percentage of coat fusion present at that particular mass (based upon the analysis of fusion proteins separated by PAGE and stained with Coomassie blue).
**Mass is corrected for sodium ions (23 Da).

TABLE 4
Proteolytic Degradation Profiles

| TMV149 | MW (Da) |
|---|---|
| GSDGAVQPDGGQPAVSYSITTPSQ-(SEQ ID NO: 39) | 18,816.5 |
| SDGAVQPDGGQPAVSYSITTPSQ-(SEQ ID NO: 40) | 18,759.5 |
| GAVQPDGGQPAVSYSITTPSQ-(SEQ ID NO: 41) | 18,557.4 |
| AVQPDGGQPAVSYSITTPSQ-(SEQ ID NO: 42) | 18,500.4 |
| VQPDGGQPAVSYSITTPSQ-(SEQ ID NO: 43) | 18,429.4 |
| QPDGGQPAVSYSITTPSQ-(SEQ ID NO: 44) | 18,330.3 |
| GGQPAVSYSITTPSQ-(SEQ ID NO: 45) | 17,990.2 |
| GQPAVSYSITTPSQ-(SEQ ID NO: 46) | 17,933.1 |
| TMV150 | MW (Da) |
| GQPDGGQPAVRNERATYSITTPSQ-(SEQ ID NO: 47) | 19,027.7 |
| NLRATYSITTPSQ-(SEQ ID NO: 48) | 17,965.1 |

Amino acids designated in bold are the natural N-terminal residues resident on TMVCP.

### Example 12

### Virion Purification and Formulation for Use in Animal Studies

PEG precipitated virion preparations (see Table 5) were resuspended in water for injection (WFI) at a concentration of 1.0 mg virus per 1.0 ml WFI. All laboratory ware used to process the virus preparations was baked at 225° C. for 18 hours. The resuspended virus preparation was solvent-extracted with chloroform and 1-butanol (8% by volume) by intermittent shaking for 1 hour at room temperature. Phases were separated by centrifugation at 10,000×G for 5 min. The aqueous phase was frozen in a dry ice/methanol bath and lyophilized overnight until dry. The lyophilized virus preparation was resuspended at a concentration of 5-10 mg virus per 1.0 ml WFI. The resuspended virus preparation was packaged in 10 ml serum vials that were sealed by crimping. Samples selection for further processing was based on both yield and percentage of fusion that remained undegraded (based on MALDI analysis).

TABLE 5
TMV Fusions Preparations Processed for Animal Studies

| TMV Fusion | Host | Extraction Method |
|---|---|---|
| TMV149 | N. benthamiana | pH-Heat, Supernatant 1 |
| TMV150 | N. benthamiana | PEI, Supernatant 1 |

### Example 13

### Vaccine Testing

The parvovirus vaccine, utilizing tobacco plant expressed TMV149 fusion and TMV150 fusion, was tested in young cats for safety and efficacy. The TMV150 fusion expressed on TMV particles proved to be safe and immunogenic by itself. TMV149 fusion vaccine was somewhat less immunogenic. Cats vaccinated with the TMV150 fusion, the TMV149 fusion or a mixture of the TMV150 fusion and the TMV149 fusion all showed significant protection against a 30% lethal dose of virulent FPV. No adjuvant was required other than what was provided by TMV proteins, some of which are known to act as superantigens (nonspecific immunostimulators). With the development and testing of this particular vaccine, the present inventors have established the usefulness and advantages of the expression system for producing common feline vaccines. The TMV149 fusion and the TMV150 fusion epitopes are the two principal hemagglutinating and neutralizing antibody-inducing antigens on the surface of FPV. The sequences of the two epitopes overlap. Cats immunized with these epitopes will develop virus neutralizing antibodies and will be partially protected against challenge with virulent virus. Therefore, cats were immunized with either TMV149 fusion or TMV150 fusion peptides, or with both, and then monitored for the vaccine's safety, immunogenicity and efficacy. Cats were immunized with 100-200 μg of each peptide, starting at 8-12 weeks of age, and with a second immunization 4 weeks later. They were then challenged orally with a large dose of virulent FPV. Both immunogens appeared completely safe, inducing no fever, depression or local reactions. Antibodies were measured using ELISA. After the second immunization, significant titers of antibodies were detected in ELISA run against whole parvovirus. Cats receiving the TMV149 fusion and the TMV150 fusion gave slightly higher responses than cats immunized with the TMV149 fusion or the TMV150 fusion. After challenge, cats immunized with the TMV150 fusion (either alone or in combination with the TMV149 fusion) appeared to be solidly protected, as evidenced by minimal signs of disease and no mortality, when compared to control cats immunized with TMV alone (that did not express the TMV150 fusion or the TMV149 fusion). It was concluded that the TMV150 fusion peptide, when delivered on TMV particles was a safe and effective vaccine, and moreover, did not require additional adjuvants.

To summarize:

1. Cats immunized with the TMV149 fusion or the TMV150 fusion (100-200 μg) made detectable antibody responses as measured by ELISA against whole FPV.

2. The antibody response to 200 μg of the TMV149 fusion or the TMV150 fusion was greater than to 100 μg.

3. Cats immunized with a combination of the TMV149 fusion and the TMV150 fusion made better antibody responses than cats immunized with either protein alone.

4. Cats vaccinated with the TMV150 fusion, or the TMV149 fusion and the TMV150 fusion, showed better protection to virulent parvovirus challenge than control cats that were unimmunized or immunized with TMV. The TMV150 fusion was more protective than the TMV149 fusion.

5. Both the TMV149 fusion and the TMV150 fusion prevented mortality; the TMV150 fusion was more effective at reducing morbidity. The TMV150 fusion-immunized cats were significantly less febrile, showed few clinical signs of illness and were markedly less leukopenic than unimmunized cats or cats immunized with control TMV.

6. Immunity conferred by the TMV150 fusion was not sterilizing, which is typical of killed parvovirus vaccines. Immunized cats showed mild signs of disease but had pronounced immunological memory.

### Example 14

### Construction of Parvo-Virus Derived Peptides Fused to the N-Terminus of the TMV U1 Coat Protein Via Methionine Linkage

Table 6 contains amino acid sequences of TMV U1 based CP fusions used or generated. The construction of the TMV150-parvo fusion is described in Example 7. The production of the TMV150 fusion virus is described in Example 9. The parvovirus epitope of interest is underlined. The TMV150 virus was modified to contain the amino acid methionine immediately preceding (TMV150/198 fusion) or following (TMV150/199 fusion) the highly conserved tyrosine (Y) residue of the TMV U1 CP. The presence of the methionine residue renders the peptide susceptible to removal by CNBr cleavage treatment.

Procedures

The modification of the TMV150 fusion virus to generate the TMV150/198 fusion and TMV150/199 fusion was performed using PCR and standard molecular biology procedures. The oligonucleotides JAL198, JAL199, and JAL200 were produced for this experiment. JAL198 (ATG TAC AGT ATC ACT ACT CCA TCT CAG) (SEQ ID NO: 49) is a forward oligonucleotide that anneals to nine codons from the 5′ end of the TMV U1 CP ORF. JAL199 (TAC ATG AGT ATC ACT ACT CCA TCT CAG) (SEQ ID NO: 50) is a forward nucleotide that mutates the 5′ end of the TMV U1 CP ORF to encode YMSITTPSQ (SEQ ID NO. 51). JAL200 (AGT AGC TCT TTC GTT TCT TAC TGC) (SEQ ID NO: 52) is a reverse oligonucleotide that anneals to the TMV150 CP fusion at nucleotides 5756-5759, the parvovirus epitope codons for AVRNERAT (SEQ ID NO: 53).

Vector Preparation

The plasmid pBTI801, which contains a full length infectious cDNA of TMV U1 under the control of the T7 RNA polymerase promoter, was digested with the restriction enzymes NcoI and PacI, which cut the TMV U1 cDNA at nucleotides 5459 and 5781, respectively. The digested DNA was phosphatased with calf alkaline phosphatase and electrophoresed through an agarose gel. The approximately 9 kb sized vector fragment was then isolated from the agarose.

Insert Preparation

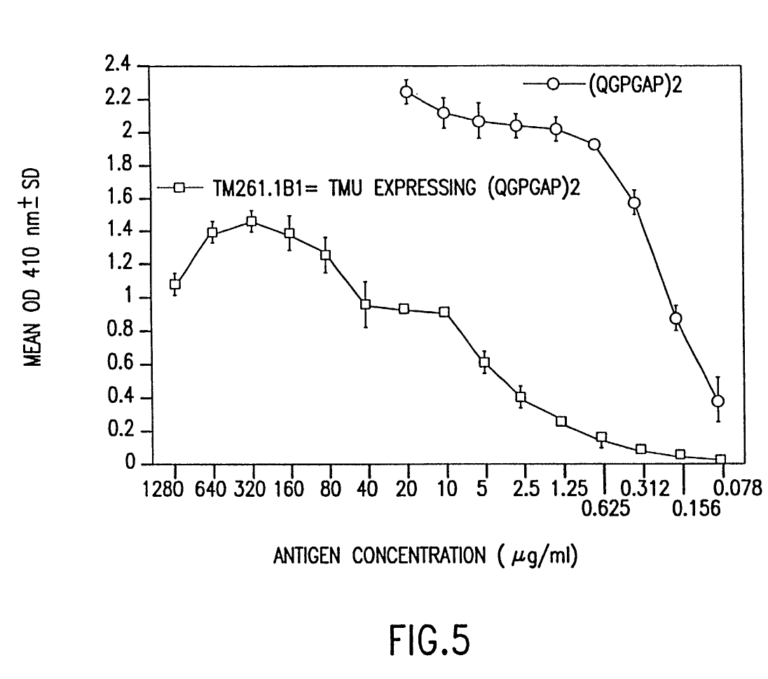

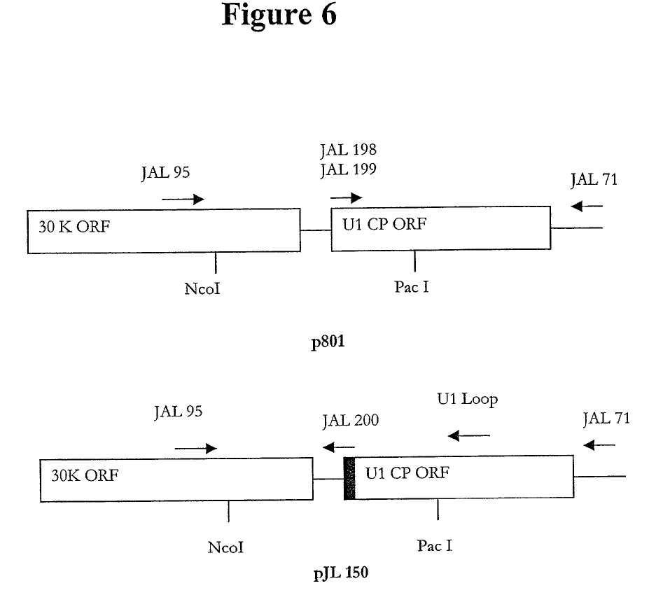

Oligonucleotides JAL198 (SEQ ID NO: 40) and JAL199 (SEQ ID NO: 41) were treated with T4 polynucleotide kinase and then used in the following PCR reactions: JAL71 (CGT CGG CCG CAC GTG TGA TTA CGG ACA CAA TCC G) (SEQ ID NO: 54) and JAL198 using pJL150 template DNA and JAL71 and JAL199 using pBTI 801 template DNA (see FIG. 6). JAL71 is a reverse oligonucleotide that anneals to TMV U1 nucleotides 6217-6240. Both PCR reactions amplify up the complete TMV CP ORF, a DNA fragment of approximately 530 bp. JAL200 and JAL95 (GTC GTC ACG GGC GAG TGG AAC TTG CCT) (SEQ ID NO: 55) were used as primers in a PCR reaction of pJL150 template DNA. JAL95 is a forward oligonucleotide that anneals to TMV U1 nucleotides 5119-5145. pJL150 is a TMV-U1 based clone containing the parvovirus epitope codons fused to the 5′ end of the U1 CP gene (see FIG. 6). Translation of this ORF generates a CP beginning with the amino acid sequence described in Table 6. The PCR product of JAL200 and JAL95 is approximately 660 bp in size. The JAL71/198 PCR product was then ligated to the JAL200/95 PCR product. Similarly, the JAL71/199 and JAL200/95 PCR products were ligated together. These ligated DNAs were used as templates for PCR reactions using the primers JAL95 and U1 loop. The U1 loop (GTC TAA TAC CGC ATT GTA C) (SEQ ID NO: 55) is a reverse oligonucleotide that anneals to the TMV U1 CP loop. The resulting PCR product was approximately 800 bp in size. Both PCR products were extracted with phenol/CHCl₃ and precipitated with ammonium acetate and ethanol after PCR, then resuspended in sterile distilled water and finally digested with the restriction enzymes PacI and NcoI. For each digested PCR product, a band of approximately 385 bp, containing the 3′ end of the U1 “30K” gene and the mutated parvovirus epitope fused to the 5′ end of the U1 CP ORF, was isolated from an agarose gel. This DNA fragment was ligated into the NcoI-PacI digested pBTI801 vector fragment, prepared as described in Example 5. The plasmids which resulted from these two ligations are named pJL150/198 or pJL150/199. Ligated DNA was transformed into E. coli and DNA amplified and prepared from individual transformed DNA colonies. The DNA was transcribed with T7 RNA polymerase in the presence of rNTPs and GpppG cap analog. The transcripts were transfected into protoplasts and the protoplasts cultured in the appropriate liquid medium. Approximately 3 days post-transfection, protoplast extracts were generated and analyzed by SDS-PAGE and Western blotting, using rabbit anti-TMV U1 sera as the primary antibody and goat anti-rabbit (alkaline phosphatase conjugate) as the secondary antibody. The results demonstrated a fusion protein of approximately the expected size was generated by TMV150/198 fusion and TMV150/199 fusion.

TABLE 6
Amino acid sequence of TMV U1 based CP fusions

| Construct name | N-terminal TMVCP amino acid sequence |
|---|---|
| Wild-type U1 CP | MYSITTPSQ- |
| (SEQ ID NO: 57) | |
| TMV150 fusion | MGQPDGGQPAVRNERATYSITTPSQ- |
| (SEQ ID NO: 58) | |
| TMV150/198 fusion | MGQPDGGQPAVRNERATMYSITTPSQ- |
| (SEQ ID NO: 59) | |
| TMV150/199 fusion | MGQPDGGQPAVRNERATYMSITTPSQ- |
| (SEQ ID NO: 60) | |

### Example 15

### Extraction and Purification of TMVCP Fusion Virions, pH-Heat Extraction

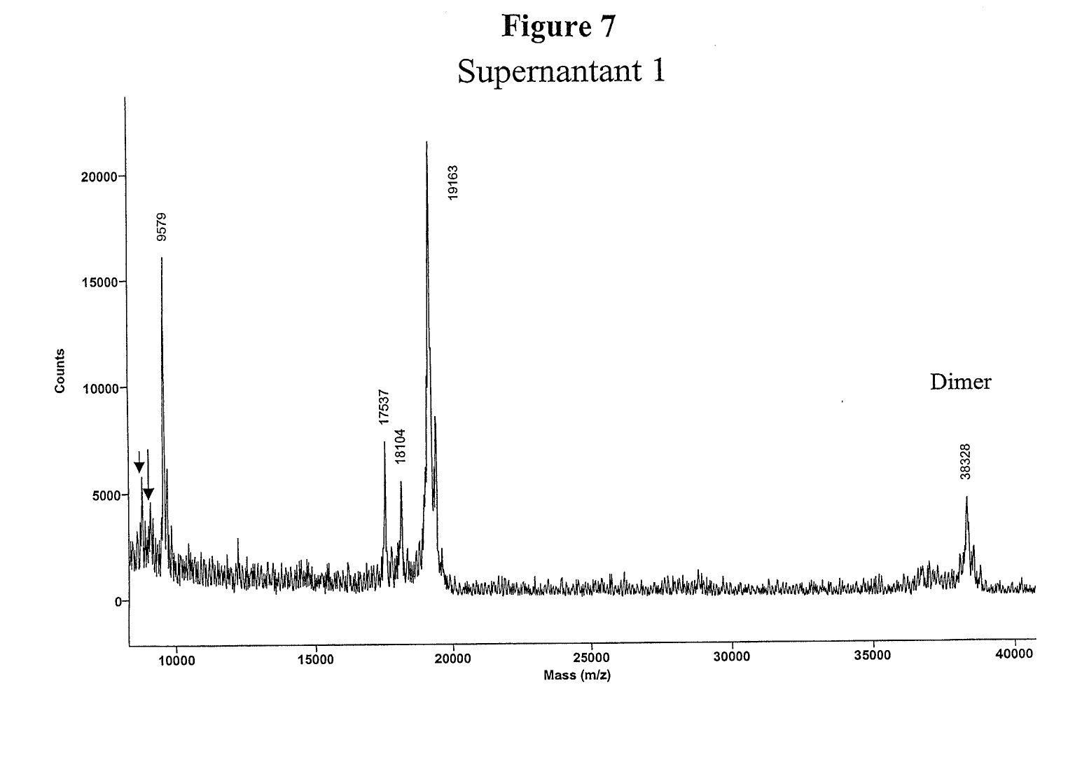

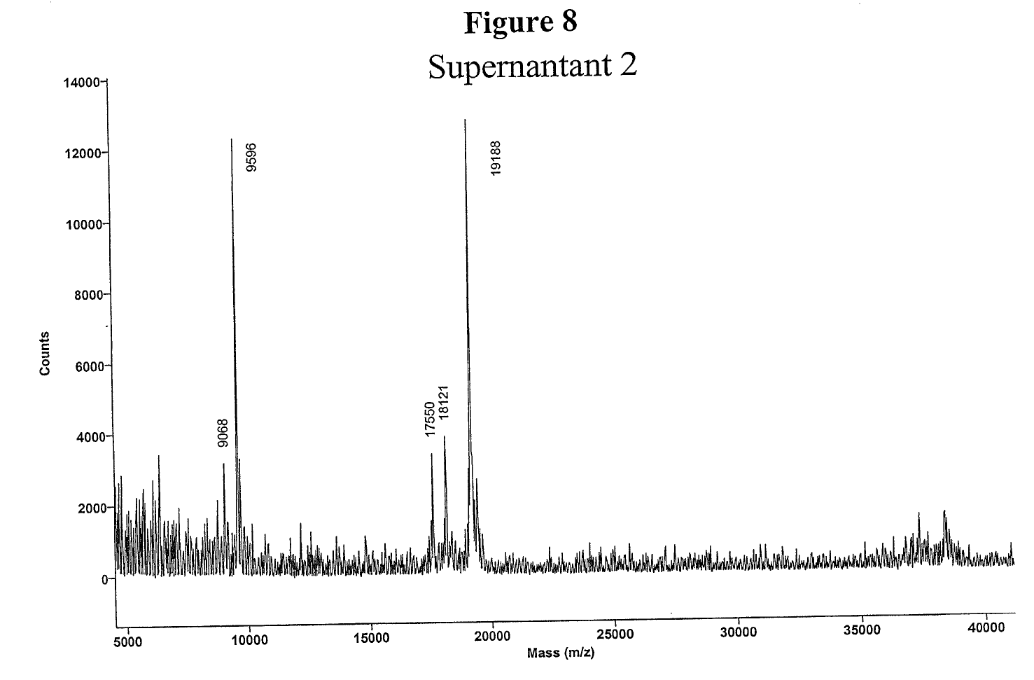

N. benthamiana, produced in a growth room, were inoculated with the TMV derivative containing a Parvo epitope fused to the N-terminus of the CP via a methionine residue (TMV150/198 and YMV150/199 fusions). Initial screening of plants inoculated with the TMV150/198 and TMV150/199 fusions indicated that the TMV150/198 virus produced a higher yield than the TMV150/199 virus. Further analysis of the TMV150/199 fusion was not pursued. Plants inoculated with the TMV150/198 fusion were harvested 2-3 weeks post inoculation after systemic spread of the virus. Leaf and stalk tissue (221 g) was macerated in a 1 L Waring blender for 2.0 min. at the high setting with 300 ml of chilled 0.04% Na₂S₂O₅. The macerated material was strained through four layers of cheese cloth to remove fibrous material to produce a “green juice”. The resultant “green juice” was adjusted to a pH of 5.0 with H₃PO₄. The pH adjusted green juice was heated to 47° C. and held at this temperature for 5 min. and then cooled to 15° C. The heat-treated green juice was centrifuged at 5,000×G for 5 min. resulting in two fractions, Supernatant 1 and Pellet 1. The Pellet 1 fraction was resuspended in distilled water using a volume of water equivalent to ½ of the initial “green juice” volume. The resuspended Pellet 1 was adjusted to a pH of 7.5 with NaOH and centrifuged at 5,000×G for 5 min. resulting in two fractions, Supernatant 2 and Pellet 2. Virus was precipitated from both Supernatant 1 and 2 fractions by the addition of PEG 6,000 and NaCl (4% by volume). After incubation at 4° C. for 1 hour, precipitated virus was recovered by centrifugation at 10,000×G for 15 min. The virus pellet was resuspended in 10 mM NaKPO₄ buffer, pH 7.2 and clarified by centrifugation at 10,000×G for 10 min. The clarified virus preparation was precipitated a second time by the addition of PEG 6,000 and NaCl (4% by volume). Precipitated virus was recovered by centrifugation as described above. PEG purified virion preparations derived from Supernatants 1 and 2 were analyzed by MALDI-TOF as described in Example 11 and mass weights determined (Table 7). Three protein masses were detected corresponding to the predicted full length TMVCP fusion, a proteolytic degradation product containing an N-terminal arginine residue and a protein containing an N-terminal methionine residue resulting from initiation of translation on an internal methionine or proteolytic degradation (see Table 8 and FIGS. 7 and 8).

TABLE 7
Product Mass Characterization

| | Product Mass |
|---|---|
| Sample | (MALDI) (Daltons) |
| Example 15, Supernatant 1, PEG2 | 19,164; 18,105; 17,537 |
| Example 15, Supernatant 2, PEG2 | 19,188; 18,122; 17551 |
| Example 16, CNBr Pellet 1 | 19,330; 17,570 |
| Example 16, CNBr, 10 Kd Permeate, | 1736, 1720, 1758, 1828 |
| Resuspended Lyophilisate | |
| Example 19, Pellet 1, HPLC, 32.5 minutes | 19,211; 17,439 |
| Example 19, Supernantant 1, HPLC, | 1737 |
| 17.4 minutes | |

TABLE 8
Coat Fusion Products.

| TMV150/198 | MW |
|---|---|
| (TMVCP amino acids are highlighted in bold) | (Daltons) |
| GQPDGGQPAVRNERATMYSITTPSQ- (SEQ ID NO: 47) | 19,159.7 |
| NERATMYSITTPSQ- (SEQ ID NO: 48) | 18,097.2 |
| MYSITTPSQ- (SEQ ID NO: 57) | 17,525.9 |

### Example 16

### Cyanogen Bromide Cleavage of TMVCP Fusions

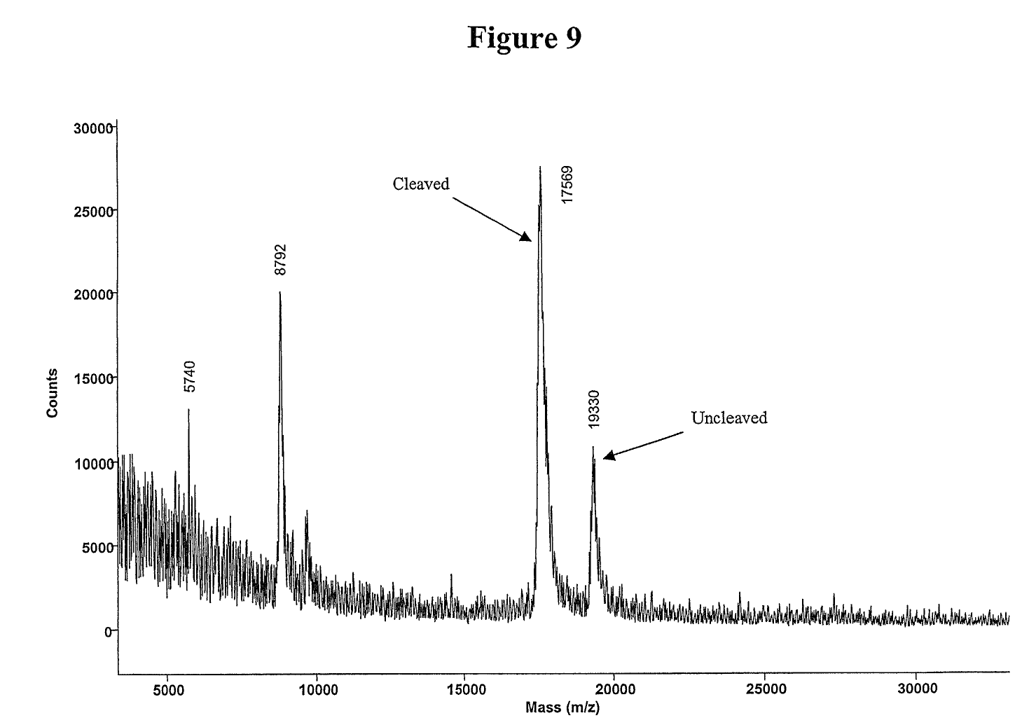

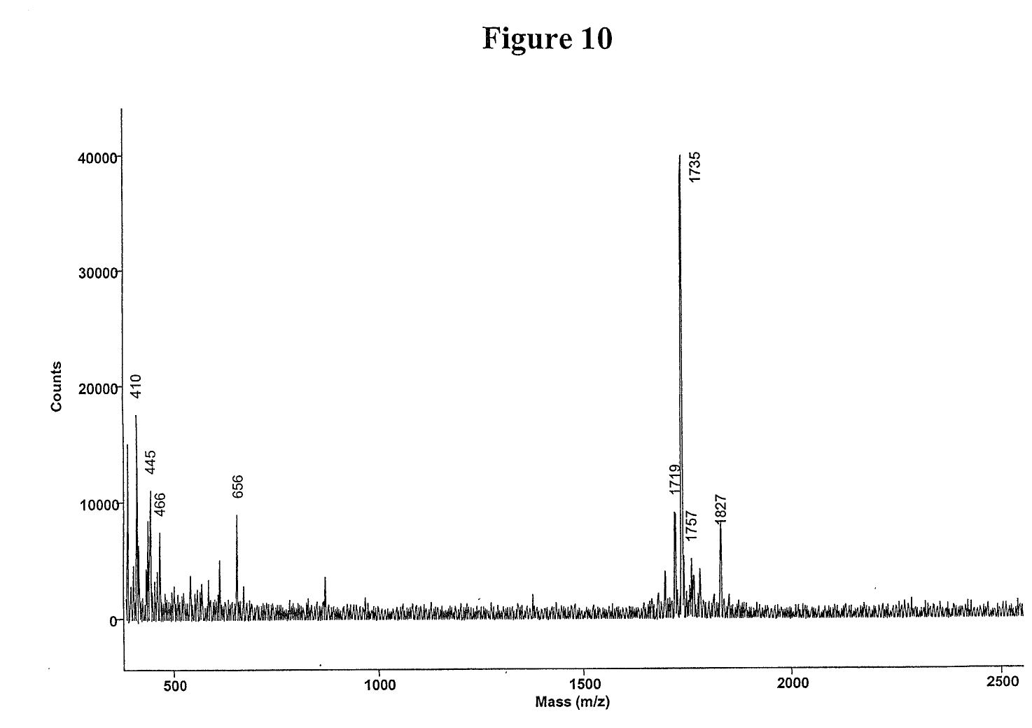

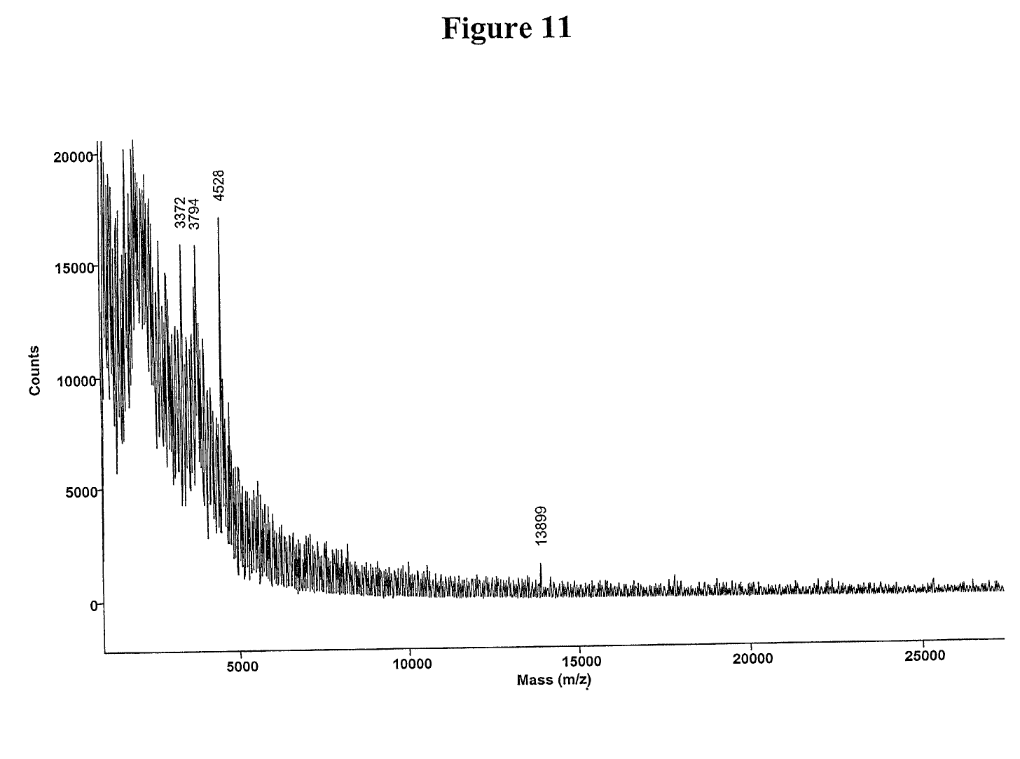

30 mg (4.4 mg virus/ml) of the purified TMV150/198 fusion (Supernatant 1-PEG 2 prepared as described in Example 15) was mixed with 21 ml of formic acid and 2.2 ml of di H₂O resulting in 1 mg/ml protein in 70% formic acid. 30 mg of solid CNBr was added to the reaction mix, dissolved by shaking, and incubated in the dark at room temperature for 6 h. After the 6 h incubation, 350 ml of di H₂O was added to the reaction mix, incubated in a dry ice-ethanol bath until frozen and lyophilized to dryness. The lyophilized powder was resuspended in 10 ml di H₂O and centrifuged at 6,000×G for 5 min. resulting in a pellet 1 and supernatant 1 fraction. The pellet 1 fraction was washed by resuspension in 10 ml di H₂O and separated by centrifugation at 6,000×G for 5 min. resulting in pellet 2 and supernatant 2 fractions. Supernatant 1 and 2 fractions were combined and filtered through a 10 Kd molecular weight cut-off Amicon centricon. The 10 Kd permeate was incubated in a dry ice-ethanol bath until frozen and lyophilized to dryness. Lyophilized material was resuspended in di H₂O for analysis. CNBr cleaved products were analyzed by MALDI-TOF as described in Example 17 and mass weights determined (Table 7). The CNBr pellet 1 contained two products with mass weights of 19,330 and 17,570 Da corresponding to uncleaved and cleaved TMVCP, respectively (FIG. 9). Both TMVCP species have an apparent increase in mass that is likely due to acid ester formation. The resuspended 10 Kd permeate lyophilisate contains predominantly a 1,736 Da species and minor quantities of 1,720; 1,758; and 1,828 Da species (FIG. 10). The 1736 fragment corresponds to the predicted mass of the released parvo peptide sequence containing a carboxy-terminal homoserine. No uncleaved or cleaved TMVCP was detected in the 10 Kd permeate lyophilisate sample (FIG. 11).

### Example 17

### Analysis of CP Fusions and CNBr Cleaved Fusions by MALDI

MALDI-TOF (sinapinic acid) analysis of products with masses above 5 kDa. Varying concentrations of each sample were diluted 1:1 with sinapinic acid (Aldrich, Milwaukee, Wis.) matrix, 1 μL was applied to a stainless steel MALDI plate surface and allowed to air dry for analysis. The sinapinic acid was prepared at a concentration of 10 mg/ml in 0.1% aqueous TFA/acetonitrile (70/30 by volume). MALDI-TOF mass spectra were obtained with a PerSeptive Biosystems Voyager DE-PRO (Houston, Tex.) operated in the linear mode. A pulsed nitrogen laser operating at 337 nm was used in the delayed extraction mode for ionization. An acceleration voltage of 25 kV with a 90% grid voltage and a 0.1% guide wire voltage was used. Approximately 100 scans were acquired and averaged over the mass range of 2-156 kDa. with a low mass gate of 2000. Ion source and mirror pressures were approximately 5×10⁻⁸ and 3×10⁻⁸ Torr, respectively. All spectra were mass calibrated with a single-point fit using horse apomyoglobin (16,952 Da).

MALDI-TOF (α-cyano-4-hydroxycinnamic Acid) Analysis of Products with Masses Below 5 kDa

Varying concentrations of each sample were diluted 1:1 with recrystallized α-cyano-4-hydroxycinnamic acid (CHCA) (Aldrich, Milwaukee, Wis. matrix, 1 μl was applied to a stainless steel MALDI plate surface and allowed to air dry for analysis. The CHCA was prepared at a concentration of 10 mg/ml in 0.1% aqueous TFA/acetonitrile/ethanol (1:1:1 by volume). MALDI-TOF mass spectra were obtained with a PerSeptive Biosystems Voyager DE-PRO (Houston, Tex.) operated in the reflectron mode. A pulsed nitrogen laser operating at 337 nm was used in the delayed extraction mode for ionization. An acceleration voltage of 20 kV with a 74% grid voltage and a 0.05% guide wire voltage was used. About 100 scans were acquired and averaged over the mass range of 385-8500 Da. with a low mass gate of 350. Ion source and mirror pressures were approximately 5.9×10⁻⁸ and 2.8×10⁻⁸ Torr, respectively. All spectra were mass calibrated with a single point fit using Angiotensin I (1,297.51 Da).

### Example 18

### N-Terminal Amino Acid Sequence Analysis of CNBr Cleaved and Purified Peptide

N-terminal amino acid sequencing of the resuspended 10 Kd permeate lyophilisate containing the CNBr-released peptide was performed by the University of Michigan Medical School Protein and Carbohydrate Structure Facility. The lyophilized 10 Kd permeate was resuspended in di H₂O at a protein concentration of 2.6 mg protein/ml and sequenced using an automated ABI Model 477 sequenator. The procedure employed standard Edman degradation to sequentially cleave and identify amino acids starting at the amino terminus (N-terminus) of the peptide. The instrument is capable of detecting all 20 common amino acids, as well as several modified forms. It was operated in a liquid-pulse mode. Sequencing was carried out 15 cycles to identify the first 15 amino acids of the peptide. After 13 cycles, the repetitive yield dropped below an about that allowed calling residues 14 and 15 with confidence. The identity of peptides was confirmed by matching the first 13 amino acids of the predicted sequences of each peptide.

### Example 19

### Separation and Purification of CNBr Cleaved Peptide by HPLC

Pellet 1 and Supernatant 1 fractions of CNBr cleaved TMV150/198 fusion, as described in Example 16, were separated by HPLC and peak fractions analyzed by MALDI-TOF as described in Example 17. HPLC Separation was performed on a Hewlett-Packard (Agilent Technologies) Model 1100 HPLC with photo diode array detection capabilities. The conditions were as follows:

| | |
|---|---|
| Column: | 0.2 × 250 Vydac Narrowbore 219TP52 Diphenyl |
| | Reverse Phase Column, 5 μm particle size |
| Flow Rate: | 0.25 mL/min. |
| Solvent A: | 5% Acetonitrile, 0.1% TFA (0.22 μm-filtered |
| | before use) |
| Solvent B: | 95% Acetonitrile, 0.1% TFA, (0.22 μm-filtered |
| | before use) |
| Gradient: | Isocratic 5 min. Solvent A, then to 100% Solvent B |
| | over 40 min.; held 5 min. at 100% Solvent B; 5 min. |
| | then return to initial conditions. |
| Detection: | UV absorbance at 210 nm and 280 nm simultaneously |
| Injection Volume: | 100 μL (containing about 3 μg total protein by |
| | BCA assay) |
| Temperature: | Not controlled (ambient conditions) |

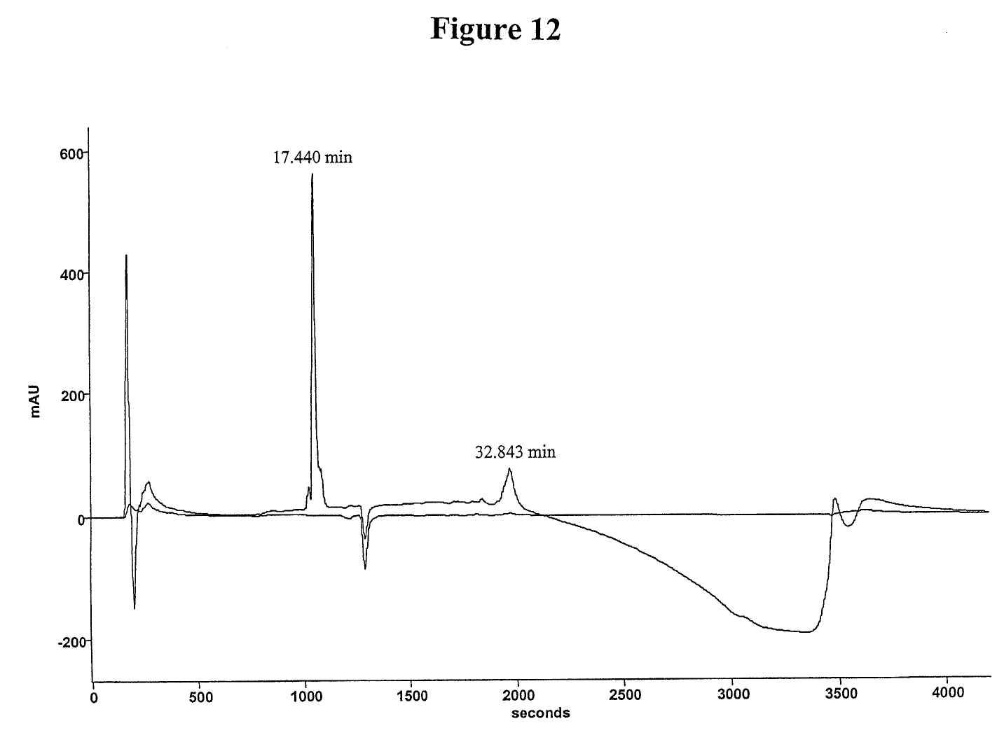

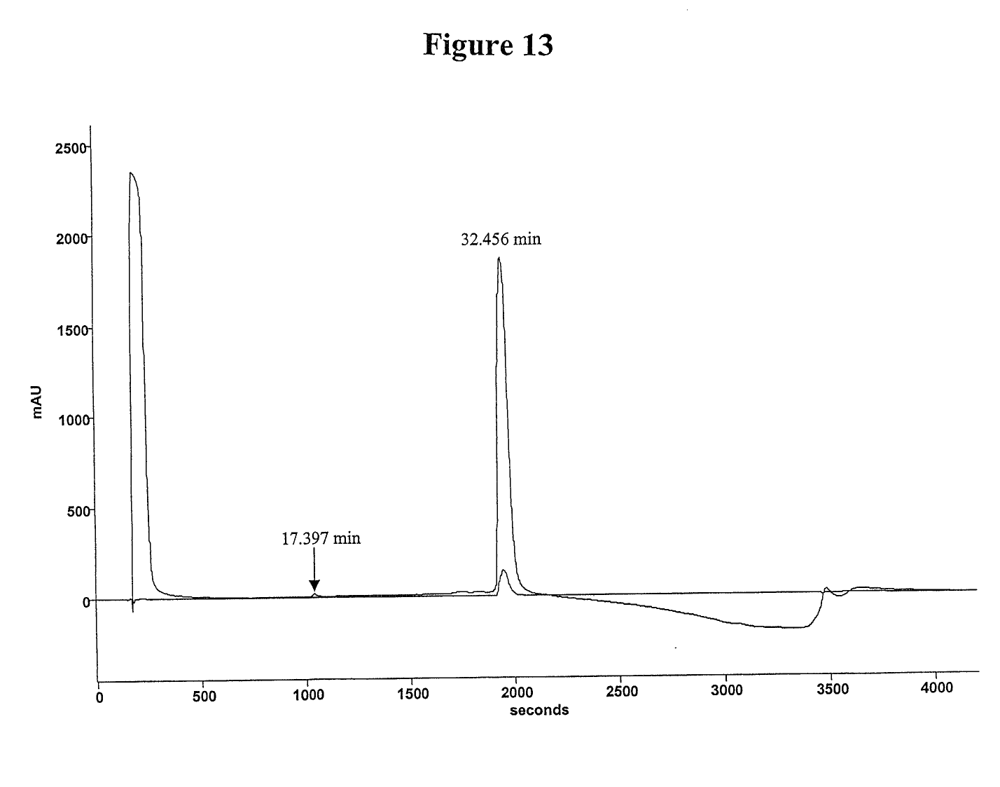

The HPLC chromatograms for the separations of the Pellet 1 and Supernatant 1 fractions are shown in FIGS. 12 and 13, respectively. The major peaks detected for each sample (17.4 min.—Supernatant 1 and 32.5 min.—Pellet 1) were collected and analyzed by MALDI (see Table 7). HPLC effectively separated cleaved peptide, eluting at 17.4 min., from uncleaved and cleaved TMVCP that eluted at 32.5 min.

Although the invention has been described with reference to the presently preferred embodiments, it should be understood that various modifications can be made without departing from the spirit of the invention.

All publications, patents, patent applications, and web sites are herein incorporated by reference in their entirety to the same extent as if each individual publication, patent, patent application, or web site was specifically and individually indicated to be incorporated by reference in its entirety.

### LITERATURE CITED

Ahlquist, P. G., and French, R. C. 1986. RNA transformation vector. European Patent Appl. 194,809.

Bruening, G., 1978. Comovirus group, C.M.I./A.A.B. Descriptions of plant viruses, No. 199. Wm. Culross and Son Ltd., Coupar Angus, Perthshire, Scotland.

Butler, P. J. G., Mayo, M. A. 1987. Molecular architecture and assembly of tobacco mosaic virus particles, The molecular biology of the positive strand RNA viruses. (D. J. Rowlands, M. A. Mayo, and B. W. J. Mahy, eds.), Academic Press, London. pp. 237-57.

Charoenvit, Y., Collins, W. E., Jones, T. R., Millet, P., Yuan, L., Beaudoin, R. L., Broderson, J. R., and Hoffman, S. L. 1991a. Inability of malaria vaccine to induce antibodies to a protective epitope within its sequence. Science 251:668-71.

Charoenvit, Y., Mellouk, S., Cole, C., Bechara, R., Leef, M. F., Sedegah, M., Yuan, L., Robey, F. A., Beaudoin, R. L., and Hoffman, S. L. 1991b. Monoclonal, but not polyclonal, antibodies protect against Plasmodium yoelii sporozoites. J. Immunol. 146:1020-5.

Dawson, W. O., Beck, D. L., Knorr, D. A., and Grantham, G. L. 1986. cDNA cloning of the complete genome of tobacco mosaic virus and production of infectious transcripts. Proc. Natl. Acad. Sci. USA 83:1832-6.

Dawson, W. O., Bubrick, P., and Grantham, G. L. 1988. Modifications of the tobacco mosaic virus coat protein gene affecting replication, movement, and symptomatology. Phytopathol. 78:783-9.

Dawson, W. O., Lewandowski, D. J., Hilf, M. E., Bubrick, P., Raffo, A. J., Shaw, J. J., Grantham, G. L., and Desjardins, P. R. 1989. A tobacco mosaic virus-hybrid expresses and loses an added gene. Virol. 172:285-92.

Donson, J., Kearney, C. M., Hilf, M. E., and Dawson, W. O. 1991. Systemic expression of a bacterial gene by a tobacco mosaic virus-based vector. Proc. Natl. Acad. Sci. USA 88:7204-8.

Donson, J., Dawson, W. O., Grantham, G. L., Turpen, T. H., Turpen, A. M., Garger, S. J., and Grill, L. K. 1992. Recombinant viral vectors having heterologous subgenomic promoters for systemic expression of foreign genes. U.S. Pat. No. 5,316,931.

French, R., Janda, M., and Ahlquist, P. 1986. Bacterial gene inserted in an engineered RNA virus: Efficient expression in monocotyledonous plant cells. Science 231:1294-7.

Gibbs, A. J. 1977. Tobamovirus group, C.M.I./A.A.B. Descriptions of plant viruses, No. 184. Wm. Culross and Son Ltd., Coupar Angus, Perthshire, Scotland.

Goelet, P., Lomonossoff, G. P., Butler P. J. G., Akam, M. E., and Karn, J. 1982. Nucleotide sequence of tobacco mosaic virus RNA. Proc. Natl. Acad. Sci. USA 79:5818-22.

Gooding, Jr., G. V., and Hebert, T. T. 1967. A simple technique for purification of tobacco mosaic virus in large quantities. Phytopathol. 57:1285.

Hamamoto, H., Hashida, E., Matsunaga, Y., Nakagawa, N., Nakanishi, N., Okada, Y., Sugiyama, Y., and Tsuchimoto, S. 1993a. Plant virus vector for foreign gene expression—contains foreign gene down stream of viral coat protein gene, linked by read-through sequence. PCT Patent Application WO 93/JP408.

Hamamoto, H., Sugiyama, Y., Nakagawa, N., Hashida, E., Matsunaga, Y., Takemoto, S., Watanabe Y., and Okada, Y. 1993b. A new tobacco mosaic virus vector and its use for the systemic production of angiotensin-I-converting enzyme inhibitor in transgenic tobacco and tomato. Bio/Technol. 11:930-2.

Haynes, J. R., Cunningham, J., von Seefried, A., Lennick, M., Garvin, R. T., and Shen, S. -H. 1986. Development of a genetically-engineered, candidate polio vaccine employing the self-assembling properties of the tobacco mosaic virus coat protein. Bio/Technol. 4:637-41.

James, E. A., Garvin, R. T., and Haynes, J. R. 1985. Multispecific immunogenic proteins. European Patent Application, 174,759.

Krebbers, E., Bosch, D., and Vandekerckhove, J. 1992. Prospects and progress in the production of foreign proteins and peptides in plants, Plant Protein Engineering. (P. R. Shewry and S. Gutteridge, eds.), Cambridge University Press, Cambridge. pp. 316-24.

Kumagai, M. H., Turpen, T. H., Weinzettl, N., della-Cioppa, G., Turpen, A. M., Donson, J., Hilf, M. E., Grantham, G. L., Dawson, W. O., Chow, T. P., Piatak Jr., M., and Grill, L. K. 1993. Rapid, high level expression of biologically active α-trichosanthin in transfected plants by a novel RNA viral vector. Proc. Natl. Acad. Sci. USA 90:427-30.

Lomonossoff, G. P., and Johnson, J. E. 1992. Modified plant viruses as vectors. PCT Application WO 92/18618.

Mason, H. S., Lam, D. M -K., and Arntzen, C. J. 1992. Expression of hepatitis B surface antigen in transgenic plants. Proc. Natl. Acad. Sci. USA 89:11745-9.

Okada, Y., and Han, K. 1986. Plant virus RNA vector. Japanese Patent Application 61/158443.

Okada, Y., and Takamatsu, N. 1988. A plant virus RNA vector. Japanese Patent Application 63/200789.

Pelcher, L. E., Halasa, M. C. 1982. An RNA plant virus vector or portion thereof, a method of construction thereof, and a method of producing a gene derived product therefrom. European Patent Appl. 067,553.

Pelham, H. R. B. 1978. Leaky UAG termination codon in tobacco mosaic virus RNA. Nature 272:469-71.

Skuzeski, J. M., Nichols, L. M., Gesteland, R. F., and Atkins, J. F. 1991. The signal for a leaky UAG stop codon in several plant viruses includes the two downstream codons. J. Mol. Biol. 218:365-73.

Takamatsu, N., Ishikawa, M., Meshi, T., and Okada, Y. 1987. Expression of bacterial chloramphenicol acetyltransferase gene in tobacco plants mediated by TMV-RNA. EMBO J. 6:307-11.

Takamatsu, N., Watanabe, Y., Yanagi, H., Meshi, T., Shiba, T., and Okada, Y. 1990. Production of enkephalin in tobacco protoplasts using tobacco mosaic virus RNA vector. FEBS Lett. 269:73-6.

Turpen, T. H., and Grill, L. K. Apr. 4, 1989. New products through viral coat protein modification. Biosource Genetics Corporation, Record of Invention, First Written Disclosure.

Usha, R., Rohll, J. B., Spall, V. E., Shanks, M., Maule, A. J., Johnson, J. E., and Lomonossoff, G. P. 1993. Expression of an animal virus antigenic site on the surface of a plant virus particle. Virol. 197:366-74.

van Kammen, A., and de Jager, C. P. 1978. Cowpea mosaic virus, C.M.I./A.A.B. Descriptions of plant viruses, No. 197. Wm. Culross and Son Ltd., Coupar Angus, Perthshire, Scotland.

Weiss, W. R., Berzofsky, J. A., Houghten, R. A., Sedegah, M., Hollindale, M., and Hoffman, S. L. 1992. A T cell clone directed at the circumsporozoite protein which protects mice against both Plasmodium yoelii and Plasmodium berghei. J. Immunol. 149:2103-9.

Zaitlin, M., and Israel, H. W. 1975. Tobacco mosaic virus (type strain), C.M.I./A.A.B. Descriptions of plant viruses, No. 151. Wm. Culross and Son Ltd., Coupar Angus, Perthshire, Scotland.

What is claimed is:

1. A polynucleotide encoding a fusion protein, wherein the fusion protein comprises a protein of interest linked to the N-terminus of a plant viral coat protein via a linking element, wherein the linking element is capable of being cleaved by a chemical reagent, wherein the linking element comprises a methionine and the chemical reagent is cyanogen bromide.

2. The polynucleotide according to claim 1 wherein the fusion protein is capable of being expressed in a plant or a plant cell.

3. The polynucleotide according to claim 1 wherein the protein of interest is an antigen.

4. The polynucleotide according to claim 1 wherein the plant viral coat protein is obtained from a single-stranded plus-sense RNA virus.

5. The polynucleotide according to claim 4 wherein the single-stranded plus-sense RNA virus is a tobacco mosaic virus.

6. The polynucleotide according to claim 1 wherein the protein of interest is a vaccine.

7. The polynucleotide according to claim 1 wherein the protein of interest is more than 15 amino acids long.

8. The polynucleotide according to claim 1 wherein the fusion protein is capable of being expressed and the plant viral coat protein is capable of being expressed in a form that is not linked to the protein of interest.

9. A recombinant viral nucleic acid comprising a polynucleotide according to claim 1.

10. A recombinant virus particle comprising a recombinant viral nucleic acid according to claim 9.

11. A recombinant plant virus wherein the plant viral coat protein is encoded by the polynucleotide according to claim 1.

12. A plant cell comprising the polynucleotide according to claim 1.

13. A plant cell comprising the recombinant viral nucleic acid according to claim 9.

14. A plant cell comprising the recombinant virus particle according to claim 10.

15. A plant cell comprising the recombinant plant virus according to claim 11.

16. A plant comprising the polynucleotide according to claim 1.

17. A plant comprising the recombinant viral nucleic acid according to claim 9.

18. A plant comprising the recombinant virus particle according to claim 10.

19. A plant comprising the recombinant plant virus according to claim 11.

20. A method for synthesizing a protein of interest, comprising the steps of:
(a) contacting a plant or a plant cell with a recombinant plant virus nucleic acid comprising a polynucleotide encoding a fusion protein, wherein the fusion protein comprises a protein of interest linked to the N-terminus of a plant viral coat protein via a linking element capable of cleavage by a cyanogen bromide, wherein the linking element comprises a methionine,
(b) growing the plant or the plant cell under conditions such that the fusion protein is expressed, and
(c) reacting the linking element with a chemical reagent such that at least one covalent bond between the protein of interest and the plant viral coat protein is broken.

21. The method according to claim 20 further comprising the step of:
(d) purifying the protein of interest from the plant viral coat protein.

23. The method according to claim 20 further comprising the step of:
(d) purifying the fusion protein from the plant or the plant cell, wherein step (d) is prior (c).
